SEQUENCE LISTING (9 sequences)

SEQ ID NO: 1 - DNA, 26 bp, Artificial Sequence
  pVAX1-F primer
ggagatctgcgttacataacttacgg

SEQ ID NO: 2 - DNA, 17 bp, Artificial Sequence
  pVAX1-R primer
tagaagccatagagccc

SEQ ID NO: 3 - DNA, 21 bp, Artificial Sequence
  Gfpm3-F1 primer
atgcgtaaaggagaagaactt

SEQ ID NO: 4 - DNA, 32 bp, Artificial Sequence
  Gfpm3-Rx primer
tagctctagattattatttgtatagttcatcc

SEQ ID NO: 5 - DNA, 24 bp, Artificial Sequence
  Nectin-1 forward primer
agatgtgaagctcacgtgcaaagc

SEQ ID NO: 6 - DNA, 24 bp, Artificial Sequence
  Nectin-1 reverse primer
ttggtggcctcacagatgtaggtt

SEQ ID NO: 7 - protein, 12 aa, Artificial Sequence
  Dendritic cell targeting peptide
FYPSYHSTPQRP

SEQ ID NO: 8 - DNA, 4448 bp, Artificial Sequence
  Shuttle vector pLKV1
atctatcgatacaaattcctcgtaggcgctcgggacccctatctagcgaacttttagaaaagatataaaacatcagagtatggacagttgcggatgtacttcagaaaagattagatgtctaaaaagcttgtagttaaagctttttagacatctaaatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataagcgtaccggttccaattttttcgcagtttaacttttccgacgcatttatcgtcttggtagtaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgatgcatgccatggtacccgactctagaggatctgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgcaattgtagcctgattgcccttcaccgcctggccctgagagagttgcagcaagcggtcccaattcatgaaaaatgaagaacgaaaaacaggcaatgtaattatccctgaaagtaaattaaatgcagtgattaaaagctatgaagagctatataatgcgaatgaaaagctaaaaaaatatgcagaaactgatttacctaaacggattgactatgtaaaagaaaaatacaaggaagtagtgcgtgattacaatgatttggcagatagatttaataataatttagagaaaattgattgcttggagaaagagaataaatcgcttaaaaacgaaattaagggcatttacaaagggtttaatcagtttatggaaaatacccttggtgcaactgttggacaagctaaaaagctcatgaataaccttgtgagcgaaattaaagagtttgtaaaaggtggagattttgaaaaaattcatagaaatgaaactagaacacgagatagagatgaattaagtcgttagaagctcatatttgcgttctaagacgttttaaatcatatcgagtatatttacacgtgattattacttttcgtcggcagaagcgttaaaaatggctgtaaataataaagacaagggggaacgaacgtgagcaacggtttacccttgtctttttattttaggcatttagcttttgctgagaggaaaaagtaattacgtgataaagctacttttcagaggcatgatcaaacgcattgcgtttgtgggtgtgggcaaagccgtcttattgtttttaggaattgggaagcaaactgggggggagattttgaaaaaatcgaccaacttctggggggcactacgacaccccccaggagtgcaatgtgcaatgtgaaaaaaattagaaacgttgttatatcaatgtttatagctataataacgtttctaaaaaaattaatagtattctattttttgtataatctctagacttatacagaataatagagtattatatatttataaattagaacggggagatggatatggaaagagtaaaggttggtataactttgactgaagatacgttagctagactagaagaaatttgtaaagaaatggggctaagtaaatctcaagctttatctatgttggttaataaagagtatttagaaaaaatatcaagatgaaaataaggtttttaaggcataaaaaaatcgccactggtaatggcgaaataactaacggtataggagtagtataacatgacgaagaaaaaatatgaatatgaaaagggtcgtgactggacgtttattgtatatccagaaagcgcaccagacaattggagaactgtattggatgaaactcatttactttggattgaaagtcctttgcatgataaagatatgaatgcagatggtgaaataaaaaaatctcattatcatattttattaacttttgatggtccagttactgaaaaacaagtgattaagttgattgatccattaaatacaccacttctaaaaaagttgggttgtccacgttggtttagtgtcgttatatggcacatttggacaaccctgaaaaatatcaatactcaaagagatgagattgttgggcattgtggtgcagatgtggagagttattttgaattaactaaaacaagcaaaatgagcgttatgaagacgattatcacttatatttacgaaataagattgataattatgctgatttttaatgatttgtattcaaacattctgatgattggtttgacgtaccgattaactacaacaacgttagcaatcaataagatgattgacggtatgtggttaaaaaagaaaaatgaacttagataataaaagccccctgacgaaagtcgaagggggtttttattttggtttgaggtttgcatacctactcgag

SEQ ID NO: 9 - DNA, 3853 bp, Artificial Sequence
  pNZ8150-gfp
agatctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcagtatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataataatctagagagctcaagctttctttgaaccaaaattagaaaaccaaggcttgaaacgttcaattgaaatggcaattaaacaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaatatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgacggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccttggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctcc

(19) United States
(12) Patent Application Publication
(10) Pub. No.: US 2013/0078275 A1
(43) Pub. Date: Mar. 28, 2013

(54) NOVEL SYSTEMS, VECTORS, AND METHODS FOR DELIVERY OF BIOMOLECULES TO EUKARYOTIC CELLS

(71) Applicant: Lin Tao, Chicago, IL (US)

(72) Inventor: Lin Tao, Chicago, IL (US)

(73) Assignee: The Board of Trustees of the University of Illinois, Urbana, IL (US)

(21) Appl. No.: 13/699,593

(22) PCT Filed: May 25, 2011
(86) PCT No.: PCT/US2011/038002

§ 371 Date: Nov. 21, 2012

Related U.S. Application Data
(60) Provisional application No. 61/348,082, filed on May 25, 2010.
(60) Provisional application No. 61/372,989, filed on Aug. 12, 2010.
(60) Provisional application No. 61/441,357, filed on Feb. 10, 2011.

(51) Int. Cl. A61K 47/46

(52) U.S. Cl. CPC A61K 47/46
USPC 424/204.1; 424/184.1; 424/93.4; 435/173.6; 435/375; 435/252.1; 435/320.1

(57) ABSTRACT

Embodiments described herein provide novel bacterial-based methods, systems, and delivery vehicles capable of delivering DNA, RNA, proteins, and other cargo into targeted mammalian cells, both in vitro and in vivo, with high efficiency. Delivery vehicles may be used to deliver molecules such as prophylactic or therapeutic proteins, DNA, shRNA, DNA vaccines, mucosal vaccines, modified viruses or viral components, and other bioactive molecules. Potential applications include gene therapy, wound healing therapies, cancer therapy, immune modulation, and research applications for which delivery of DNA, RNA, proteins, or other cargo into mammalian cells and tissues is required.

### CROSS REFERENCE TO RELATED APPLICATIONS

The present application claims priority to U.S. Provisional Application No. 61/348,082, filed May 25, 2010, entitled “Highly efficient delivery of therapeutic and prophylactic molecules into mammalian cells by food-grade/commensal bacteria”; U.S. Provisional Application No. 61/372,989, filed Aug. 12, 2010, entitled “Methods for RNA Interference (RNAi) Delivery System by non-invasive, food-grade and/or commensal bacteria”; and U.S. Provisional Application No. 61/441,357, filed Feb. 10, 2011, entitled “Food/commensal bacteria-based mucosal vaccine delivery system,” the entire disclosures of which are hereby incorporated by reference in their entireties.

### GOVERNMENT INTERESTS

This invention was made with Government support under Grant/Contract No. AI 066709 awarded by the National Institutes of Health. The Government has certain rights in the invention.

### TECHNICAL FIELD

Embodiments herein relate to molecular delivery systems, and, more specifically, to the delivery of molecules into eukaryotic cells by non-invasive bacterial delivery vehicles (BDVs).

### BACKGROUND

There has been increasing interest in the use of bacteria to deliver DNA molecules as vaccines or gene therapies to mammalian cells. Previously, it was hypothesized that bacteria-mediated DNA delivery into eukaryotic cells requires invasion or infection by the bacterial carrier. As a result, attenuated pathogenic bacteria such as Listeria, Salmonella, Shigella, and Yersinia have been used as DNA-delivery bacterial vectors (Detmer et al., Microbial Cell Factories 5:23, 2006; Vassaux et al., J. Pathol. 208: 290-298, 2006). These bacteria can invade mammalian cells and deliver eukaryotic expression vectors, resulting in cellular expression of the gene of interest and subsequent antigen presentation. However, a possible reversion to a virulent phenotype may occur in these attenuated bacteria.

For safety reasons, noninvasive bacteria such as food and commensal lactic acid bacteria (LABs) have been used as DNA delivery vehicles to develop DNA vaccines (Wells et al., Nat Rev Microbiol. 6:349-362, 2008). LAB are a group of Gram-positive, non-sporulating bacteria that includes species of Lactobacillus, Lactococcus, Leuconostoc, Pediococcus and Streptococcus, which produce a common metabolic end product, lactic acid, from the fermentation of carbohydrates. Food LAB are species and strains used in food- and feed-fermentation processes, and commensal LAB are those that inhabit the oral cavity, gastrointestinal and genital tracts, and other sites in vertebrates.

However, the DNA delivery efficiency with these bacteria has been low (Guimarães et al., Appl. Environ. Microbiol. 72:7091-7097, 2006). To improve DNA delivery into mammalian cells, invasive genes from pathogenic bacteria, such as the internalin gene inIA from Listeria monocytogenes (Guimarães et al., Microbes Infect. 7:836-844, 2005; Guimarães et al., Genet. Vaccines Ther. 7:4, 2009), have been cloned into the noninvasive food bacterium Lactococcus lactis. Although carrying an invasive gene significantly increased the efficiency of DNA delivery from L. lactis to mammalian cells, the success rate of DNA deliver is still low, only about 1% (Guimarães et al., 2006; Guimarães et al., 2005). Moreover, by expressing an invasive protein of a pathogenic bacterial origin, the previous noninvasive bacterium becomes invasive, which could render the LAB strain unsafe for human use.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIGS. 1A and 1B illustrate examples of a shuttle plasmid vector (pLKV1; FIG. 1A), a protein expression reporter plasmid (pNZ8150-gfp; FIG. 1B), and a DNA delivery reporter plasmid (pLKV-Red2; FIG. 1A).

FIGS. 2A and 2B show delivery of the red fluorescent reporter plasmid pLKV-Red2 into Caco-2 cells by Streptococcus gordonii V288 with (FIG. 2A) and without (FIG. 2B) penicillin and lysozyme treatment.

FIGS. 3A and 3B show Caco-2 cell uptake of GFPmut3*-expressing Lactococcus lactis NZ3900-pNZ8150-gfp with (FIG. 3A) and without (FIG. 3B) glycine treatment to weaken the bacterial cell wall. The phase contrast and fluorescent images were superimposed so that the unstained Caco-2 cells can be visualized.

FIGS. 4A-4C illustrate nisin dose-dependent GFP expression in L. lactis NZ3900, ATCC11454 (FIG. 4A, 0.8%; FIG. 4B, 1.6%; and FIG. 4C, 3.2% (v/v) nisin;

FIG. 5 illustrates GFP expression in L. murinus KC47b (Arrow A indicates low GFP expression, cell size normal; Arrow B indicates high GFP expression, cell swollen).

FIGS. 6A and 6B illustrate the conventional tkRNAi plasmid TRIP (FIG. 6A) and the novel NtkRNAi plasmid NIP1 (FIG. 6B).

FIG. 7 illustrates RNA transcription to shRNA in bacterium and processing of the shRNA to functional siRNA within the eukaryotic cell.

FIG. 8 illustrates the NbmRNAi plasmid NIP2 (FIG. 8A) and reporter plasmid NIP2-Red (FIG. 8B).

FIG. 9 illustrates a method for preparing, loading, and using a Gram-positive bacterial revenant for delivery of a cargo to a eukaryotic cell.

FIGS. 10A-C illustrate delivery of a red fluorescent protein plasmid to UM1 cells by L. lactis MG1363 bacterial revenants (FIG. 10A) and delivery of a green fluorescent protein plasmid (pCMV-MIR) to UM1 cells by S. gordonii V288 bacterial revenants (FIG. 10B; FIG. 10C shows the blue DAPI nuclear counterstained cells of FIG. 10B).

FIG. 11 illustrates an alternative method for preparing, loading, and using a Gram-positive bacterial revenant for delivery of a cargo to a eukaryotic cell.

FIG. 12 illustrates a method for preparing, loading, and using a Gram-negative BDV for delivery of a cargo to a eukaryotic cell.

FIG. 13 illustrates guided delivery of a vaccine to a eukaryotic cell by a BDV.

FIGS. 14A and 14B illustrate dosage-dependent expression of a chimeric virus like particle (BPV1 L1-gp41-2F5) in a food-grade vaccine strain of L. lactis (FIG. 14A shows dosage-dependent response to inducer nisin produced by L. lactis ATCC11454; FIG. 14B shows time course of expression after addition of the inducer nisin).

FIG. 15 illustrates examples of bacterial delivery vehicles and cargo for delivery to eukaryotic cells.

FIG. 16 illustrates a method for delivering a gene or other cargo into a eukaryotic cell with BDVs (pre-loading of bacterium, FIG. 16A; post-loading, FIG. 16B; pore formation and loaded BDV, FIG. 16C; gene delivery into mammalian cells, FIG. 16D).

FIG. 17 illustrates transfection of cells using Gram-positive bacterial revenants (FIG. 17A, Caco-2 cells transfected by pLKV1-Red2 via S. gordonii bacterial revenants; FIG. 17B, UM1 cells transfected with pLKV-Red2 via L. lactis bacterial revenants; FIG. 17C, UM1 cells transfected by pCMV-MIR via S. gordonii bacterial revenants) or Gram-negative bacterial revenants (FIG. 17D, Rh30 cells transfected by pLKV-Red2 via E. coli bacterial revenants).

FIG. 18 illustrates transfection of cells via Gram-positive L. lactis bacterial revenants (FIG. 18A, mouse dermal fibroblasts transfected with pLKV-Red2 loaded L. lactis bacterial revenants; FIG. 18B, chicken cerebral cortex cells transfected with pLKV-Red2 loaded L. Lactis bacterial revenants; FIG. 18C, chicken cerebellar cells transfected with pCMV-MIR loaded L. lactis bacterial revenants; Upper: bright field, Lower: Fluorescence).

FIG. 19 illustrates delivery of the Red2 gene into the tissue of a wound in a skin wound healing mouse model over days 0-5 (FIG. 19A, Day 0, control; FIG. 19B, Day 1; FIG. 19C, Day 2; FIG. 19D, Day 3; FIG. 19E, Day 4; FIG. 19F, Day 5).

FIG. 20 illustrates a method and kit for transfecting mammalian cells using BDVs.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

As used herein, a “vesicle” can be any membrane-bound compartment within a eukaryotic cell that encloses material taken up by the eukaryotic cell through endocytosis. A vesicle can be, but is not limited to, a vacuole, an endosome, a phagosome, a lysosome, or a phagolysosome.

As used herein, “endocytosis” means any process by which a eukaryotic cell engulfs a BDV or bacterial revenant. The term “endocytosis” is intended to encompass processes used by non-professional phagocytes to engulf bacterial delivery vehicles (BDVs) and bacterial revenants, in addition to phagocytosis by professional phagocytes. The terms “phagocytosis” and “endocytosis” may be used interchangeably for any such process occurring in a professional phagocyte or in another eukaryotic cell type (e.g., an epithelial cell, a mucosal cell, a neuron, a muscle cell, etc.).

As used herein, a “non-invasive bacterium” is a bacterium that lacks the ability to actively invade a host cell by penetration of a host cell membrane (e.g., a plasma membrane, a membrane of a vesicle, or a membrane of an organelle), by escape from a host cell vesicle after internalization of the bacterium by the host cell, by blocking maturation or fusion of a vesicle, by transforming or redirecting a vesicle, and/or by extended survival within a membrane-bound compartment such as a vesicle or organelle. The term “non-pathogenic bacterium” may be used in reference to a non-invasive bacterium that is a food-grade bacterium, a commensal bacterium, and/or a bacterium not considered to be a human pathogen.

As used herein, a “bacterial delivery vehicle,” “delivery vehicle,” “BDV,” and “wall-stripped bacterium” (used interchangeably throughout the present disclosure) is a bacterium that is naturally non-invasive, or has been rendered non-invasive (e.g., by genetic transformation), and has a weakened cell wall. A “weakened cell wall” is a bacterial cell wall that has been chemically disrupted (i.e., damaged or at least partially removed by application of one or more chemicals to the non-invasive bacterium), or physically disrupted (i.e., damaged or at least partially removed by application of one or more physical processes to the non-invasive bacterium), or both chemically and physically disrupted. Examples of chemicals that may be applied to weaken the cell wall of a non-invasive bacterium include antibiotics, glycine, lysozyme, detergents, and other antimicrobial substances known to disrupt bacterial cell walls. Examples of physical processes that may be applied to weaken the cell wall of a non-invasive bacterium include electroporation, pressure, and heating.

As used herein, the term “bacterial revenant” or “revenant” is a BDV with a plasma membrane that has been perforated by a physical process (e.g., electroporation) or by application of one or more chemicals (e.g., lysin or other pore-forming chemicals) to a non-invasive bacterium/BDV. The perforation of the plasma membrane causes bacterial cell death.

As used herein, the term “cargo” means one or more molecules or particles loaded into a BDV, bacterial revenant, or bacterium intended for use as a BDV/bacterial revenant, for delivery to a eukaryotic cell. The term “cargo” also includes molecules loaded into a BDV, or bacterium intended for use as a BDV, for production within the bacterium/BDV of one or more other “cargo” molecules (e.g., plasmids, shRNAs, proteins, etc.) to be delivered to a eukaryotic cell. Cargo can include, but is not limited to, one or more nucleic acids (e.g., single-stranded, double-stranded, plasmid, DNA, RNA, siRNA, shRNA, micro RNA), proteins, polymers, viruses or derivatives thereof, virus-like particles (VLPs), chimeric virus-like particles (CVLP's), nanoparticles, antigens, vaccines, and/or reporter plasmids/molecules, alone or in any combination. “Cargo” may be “pre-loaded” into a BDV or bacterium intended for use as a BDV/bacterial revenant by known methods (e.g., electroporation, chemical transformation, passive uptake) before or after physical/chemical treatment of the bacterium to weaken or otherwise disrupt the cell wall). “Cargo” may also be “post-loaded” into a bacterial revenant by adding the cargo to the bacterial revenant after perforation of the bacterial plasma membrane. In some examples, a first cargo may be pre-loaded into a BDV or bacterium intended for use as a BDV/bacterial revenant, the bacterial plasma membrane may then be perforated, and a second cargo may be subsequently post-loaded into the resulting bacterial revenant. Optionally, BDVs or bacterial revenants may be inactivated by heating or other known methods.

Delivery of proteins, DNAs and RNAs into mammalian cells is required for modern applications and treatments such as gene therapy, cancer therapy, and vaccination. An ideal molecular delivery system should be (i) safe; (ii) biocompatible; (iii) non-immunogenic; (iv) protect DNA/RNA/protein cargo, (v) small in size; (vi) cell and tissue specific; and (vii) highly efficient. However, such molecular delivery into mammalian cells has suffered low efficiency. Most existing delivery systems are used for in vitro transfection and have low efficiencies (i.e. 1-2%). Safe and highly efficient in vivo systems have yet to be developed.

The efficiency of non-viral gene delivery into eukaryotic/mammalian cells is dependent on four factors: DNA protection before entering the eukaryotic/mammalian cells; (ii) DNA delivery across cell membranes by bacteria entering the cells; (iii) DNA release in the endosome/phagosome; and (iv) DNA entering the nucleus for transcription. To promote efficiency, invasive procedures or infective agents are often used. These include gene gun, high voltage electroporation, lipofection, infective viruses (e.g., retroviruses, herpesviruses and adenoviruses) or bacteria (Listeria, Salmonella, Shigella, and Yersinia) (Wells et al., 2008), all of which are potentially unsafe.

To improve safety, a non-invasive food bacterium, Lactococcus lactis, has been used for gene delivery, but it needs to express a cloned invasion gene, inlA, from the pathogenic bacterium Listeria (Guimarães et al., 2006; Innocentin et al., Appl. Environ. Microbial. 75:4870-4878, 2009). This invasion gene allows L. lactis to enter mammalian cells at a low rate (1%).

The present disclosure provides molecular delivery methods and systems based on our surprising discovery that bacterial invasion of mammalian cells is not required for efficient gene delivery. We have discovered that the bacterial cell wall is the major barrier to delivery, and that stripping some or all of the cell wall can overcome that barrier. Once the cell wall is stripped, any cargo molecules inside the bacteria, such as proteins, nucleic acids, and vaccine components, can be delivered into the mammalian cells with high efficiency.

We determined that increased efficiency of delivery is due to increased bacterial internalization via phagocytosis of the wall-stripped bacteria by the eukaryotic cells. The wall-stripped bacteria are more readily internalized by eukaryotic cells, and the cargo molecules more readily released in the vacuoles, than untreated bacteria.

Our surprising discovery that stripping/disrupting the cell wall of non-invasive bacteria (e.g., Streptococcus gordonii, L. lactis, non-pathogenic E. coli) allows delivery of cargo within the bacteria to Caco-2 cells with 100% efficiency (see e.g., FIG. 2) provides new opportunities for the prevention and treatment of infections and other conditions. For example, the gene-delivery efficiency with our newly established, noninvasive and food-grade system is 100-fold higher than the presently best method with an invasive bacterium (Guimarães et al., 2006; Guimarães et al., 2009).

Disrupting the integrity of the bacterial cell wall may facilitate DNA release inside the eukaryotic cells. However, the critical stage affected by the bacterial cell wall disruption appears to be at the stage of internalization. The uptake of green fluorescent bacteria by Caco-2 cells was significantly enhanced by the treatment of the bacterial cells with cell wall-disrupting agents (FIG. 3). The exact mechanism for the enhanced bacterial internalization is unknown. One possibility is that bacteria with an intact cell wall may be more resistant to “non-professional” phagocytosis by the mammalian epithelial cells. Another possibility is that the surface of food or commensal bacteria may carry specific markers recognizable by the mammalian gut epithelial cells (Artis, Nat. Rev. Immunol. 8:411-420, 2008), and disrupting the bacterial cell wall may render these markers unrecognizable. Still another possibility is that the bacteria may carry phagocytosis-associated receptors embedded in the cell wall or its associated structures (Melendez et al., Biosci. Rep. 28:287-298, 2008). Disrupting the bacterial cell wall could expose such receptors and subsequently result in phagocytosis by the gut epithelium-derived Caco-2 cells.

Thus, embodiments of the present disclosure provide novel molecular delivery systems and methods in which delivery vehicles prepared from noninvasive food-grade or commensal bacteria are used to deliver cargo (e.g., nucleic acids, proteins, small molecules, virus particles, virus-like particles, nanoparticles, and/or drugs) to mammalian cells. Advantages over prior delivery systems include improved safety, efficiency, and efficacy. In some aspects, embodiments herein also provide an opportunity to monitor delivery of nucleic acids (e.g., a gene) to a eukaryotic cell.

In some embodiments, delivery vehicles may be wall-stripped Gram-negative bacteria. Such delivery vehicles may be prepared by treating the Gram-negative bacteria with one or more cell wall stripping agents in order to enhance uptake of the bacterial cells by the targeted eukaryotic cells. In one example, the cargo may be produced by the Gram-negative bacterial cell before the stripping of the cell wall. In another example, the cargo may be produced by the Gram-negative bacterial cell during and/or after the stripping of the cell wall. Alternatively, cargo may be loaded into the Gram-negative bacterial cell or delivery vehicle after wall-stripping and/or puncturing with known methods such as electroporation, glass-bead beating, heat shock, and/or the use of chemical reagents such as lysozyme and phage lysin.

In other embodiments, the delivery vehicles may be wall-stripped, membrane-perforated bacteria (“revenants”), such as Gram-positive revenants. In some examples, revenants may be prepared by treating Gram-positive bacteria with one or more wall-stripping agents and nisin, a food-grade antibiotic which forms pores in the bacterial cell membrane, to produce bacterial revenant delivery vehicles. Alternatively, revenants may be prepared by transforming Gram-positive bacteria with an inducible gene that encodes nisin, treating the bacteria with one or more wall-stripping agents, and inducing the gene to cause perforation of the bacterial cell membrane. Revenants may be harvested and stored by freezing or freeze-drying until use. Various molecules such as DNA, RNA or proteins can be loaded into the bacterial vehicles as described below for delivery to targeted eukaryotic cells.

In some examples, revenants may be prepared from lactic acid bacteria. Lactic acid bacteria offer several attractive features as live vaccine delivery vectors. First, the use of lactic acid and other noninvasive bacteria as delivery vehicles reduces or eliminates local discomfort and systemic manifestations, and avoids the risk associated with contaminated needles and the need for a professional healthcare infrastructure. In addition, lactic acid bacteria and other noninvasive bacteria offer opportunities for faster, simpler mass production of the delivery vehicles and increased ease of control of inoculation.

Embodiments also provide novel plasmids for gene delivery to eukaryotic cells. In some examples, bacterial vehicles or bacterial precursors thereof may be transformed with a plasmid and may transcribe at least a portion of the plasmid to produce a cargo (e.g., shRNA, siRNA, and/or a protein). In other examples, bacterial vehicles may be transformed with a plasmid that is transcribed by a eukaryotic cell after uptake of the bacterial vehicle by the eukaryotic cell. In still other examples, plasmids may be loaded into revenants for delivery to mammalian cells.

Compared with other methods used in gene therapies and mucosal immunizations, putative advantages to the methods, systems, and delivery vehicles described herein include safety, high efficiency, easy compliance (needle-free, gene gun free, high voltage free; infective agents free), cost-efficiency (trivial genetic engineering) and controllability by antibiotics. Potential applications of this technology include the efficient delivery of molecules, such as prophylactic/therapeutic proteins, DNA, RNA, shRNA, DNA vaccines, mucosal vaccines, modified viruses or viral components, and other bioactive molecules for purposes such as gene therapy, wound healing therapies, cancer therapy, immune modulation, and for use in research that requires delivery of DNA, RNA and/or proteins into mammalian cells and tissues.

In some aspects, the systems, methods, and delivery vehicles described herein may be used to deliver one or more nucleic acids, proteins, other molecules, and/or any combination thereof, to targeted eukaryotic cells.

In some aspects, embodiments described herein provide mucosal vaccines administered through the nasal, oral, anal/rectal, or vaginal route. The vaccines can also be delivered by direct injection into the tissue or under the skin. Such vaccines may be more effective than systemically administered vaccines in producing responses in mucosal tissues. In some examples, noninvasive food-grade or commensal bacteria may be used to deliver mucosal HIV vaccines in the form of a cream, suppository, spray, cream/emollient, or other topical formulation.

In one aspect, the present disclosure provides systems for delivery of molecules to a eukaryotic cell. The system may include a bacterial delivery vehicle (BDV) with a plasma membrane and a weakened cell wall, and a cargo disposed within the BDV. The BDV may be produced by physical or chemical disruption of an intact cell wall of a non-invasive bacterium, and the cargo may be delivered to the eukaryotic cell through endocytosis of the BDV by the eukaryotic cell. In some examples, the plasma membrane is perforated and the BDV is a bacterial revenant. The non-invasive bacterium may be used to produce RNA or protein for delivery to the eukaryotic cell before the plasma membrane is perforated. Alternatively, the non-invasive bacterium may be used to produce nucleic acids for delivery to the eukaryotic cell before the plasma membrane is perforated. The delivered nucleic acids may subsequently be transcribed by the eukaryotic cell to produce shRNA and/or siRNA. In some examples, additional cargo may be loaded into the bacterial revenant after the plasma membrane is perforated.

The non-invasive bacterium can be a commensal bacterium, a food-grade bacterium, or a non-pathogenic bacterium. In some examples, the non-invasive bacterium is a Gram-positive bacterium. In other examples, the non-invasive bacterium is a Gram-negative bacterium. The cargo may include one or more molecules produced within the non-invasive bacterium. The BDV may be produced by treatment of the non-invasive bacterium with one or more of lysozyme, penicillin, and glycine.

The cargo may include one or more molecules produced within the non-invasive bacterium. In one example, the cargo may include one or more of a nucleic acid molecule, a protein, a virus like particle, a chimeric virus like particle, a nanoparticle, and a therapeutic drug. In other examples, the cargo includes one or more molecules selected from the group consisting of a protein vaccine, a DNA vaccine, a protein-DNA dual vaccine, and a pseudovirus vaccine. In still other examples, the cargo includes one or more molecules selected from the group consisting of a siRNA molecule, a microRNA molecule, a shRNA molecule, and a plasmid. The eukaryotic cell may be cell of a target tissue, and the system may have a transfection efficiency within the target tissue of at least 90%, at least 95%, or 100%.

In another aspect, the present disclosure provides methods for delivering a cargo into a eukaryotic cell. Methods may include administering to the eukaryotic cell a BDV, wherein the cargo is enclosed within the BDV. The cargo may include one or more molecules selected from the group consisting of a nucleic acid molecule, a protein, a virus like particle, a chimeric virus like particle, a DNA vaccine, a protein vaccine, and a therapeutic drug. The cargo is delivered into the eukaryotic cell through endocytosis of the non-invasive BDV by the eukaryotic cell. The eukaryotic cell may be cell of a target tissue, and the cargo may be delivered to at least 90%, at least 95%, or 100% of the target tissue.

Methods may also include physically or chemically disrupting an intact cell wall of a non-invasive bacterium to produce the BDV. Some methods may include physically disrupting the cell wall and chemically disrupting the cell wall.

The cell wall may be physically disrupted by electroporation, mixing with glass beads, or heating of the non-invasive bacterium. The cell wall may be chemically disrupted by applying one or more chemicals to the non-invasive bacterium. At least one of the one or more of the chemicals may be selected from the group consisting of penicillin, vancomycin, a β-lactam antibiotic, a glycopeptide antibiotic, nisin, polymyxin B, polymyxin E, DL-threonine, glycine, phage lysin, and lysozyme. Optionally, the one or more chemicals may include two chemicals selected from the group consisting of penicillin, vancomycin, a β-lactam antibiotic, a glycopeptide antibiotic, nisin, polymyxin B, polymyxin E, DL-threonine, glycine, phage lysin, and lysozyme to the non-invasive bacterium.

In some examples, the BDV is a bacterial revenant and the method further includes physically or chemically perforating a plasma membrane of the non-invasive bacterium to produce a bacterial revenant. Some methods may also include loading the cargo into the non-invasive bacterium by electroporation.

The present disclosure also provides novel plasmids for transfection of eukaryotic cells and for monitoring transfection. A delivery vector plasmid vector may include, for example, an origin of replication, a kanamycin resistance marker, and a eukaryotic gene expression cassette. The delivery vector plasmid may be less than 4.5 kb in size, and the origin of replication and the kanamycin resistance marker may be expressible in both Gram-positive and Gram-negative bacteria. A reporter plasmid may include an origin of replication, a kanamycin resistance marker, a eukaryotic gene expression cassette, and a reporter sequence encoding a reporter molecule. The reporter plasmid may be less than 5.2 kb in size. The origin of replication and the kanamycin resistance marker may be expressible in both Gram-positive and Gram-negative bacteria. The reporter sequence may be expressible in eukaryotic cells and not expressible in Gram-positive bacteria or Gram-negative bacteria.

In another aspect, the present disclosure provides pharmaceutical compounds that include one or more BDVs or bacterial revenants admixed with an excipient. The excipient may be formulated for application to an epithelial or mucosal tissue. For example, the excipient may include one or more of a buffer, a stabilizer, a binder, a thickening agent, or a mucoadhesive. The pharmaceutical compound may be disposed on or within a food, an injectable liquid, a topical formulation, a spray, a lozenge, an enema, an inhalant, or a suppository.

A cargo may be disposed within the one or more BDVs. The cargo may include one or more of a nucleic acid molecule, a protein, a virus like particle, a chimeric virus like particle, a nanoparticle, or a therapeutic drug. In some examples, the cargo includes one or more molecules selected from the group consisting of a protein vaccine, a DNA vaccine, a protein-DNA dual vaccine, and a pseudovirus vaccine. In other examples, the cargo includes one or more molecules selected from the group consisting of a siRNA molecule, a microRNA molecule, a shRNA molecule, and a plasmid.

The present disclosure provides examples of kits and reagents for delivery of a cargo to a eukaryotic cell. A kit may include, for example, a control plasmid and one or more chemicals configured to weaken bacterial cell walls. Application of the one or more chemicals to a non-invasive bacterium may result in the formation of a BDV. A portion of the control plasmid may encode a reporter molecule.

Alternatively, a kit may include a plurality of bacterial revenants and a control plasmid. A portion of the control plasmid may encode a reporter molecule. The plurality of bacterial revenants may include a first group of bacterial revenants prepared from a first species of bacteria and a second group of bacterial revenants prepared from a second species of bacteria. The plurality of bacterial revenants may further include a third group of bacterial revenants prepared from a third species of bacteria different from the first and second species. Some of the bacterial revenants of the plurality may contain a copy of the control plasmid. The plurality of bacterial revenants may be supplied in lyophilized form. In some examples, the kit may further include a loading buffer.

Systems, methods, and delivery vehicles provided herein may be used to prevent or treat one or more infections or disease conditions. While HIV/HSV prevention is discussed below as one example of such an infection or disease, this example is provided merely by way of illustration. Persons skilled in the art will recognize that the systems, methods, and delivery vehicles provided herein may be used in a broad range of applications that involve the delivery of cargo/molecules into eukaryotic/mammalian cells. Examples of other applications include delivery of prophylactic/therapeutic proteins, DNA, RNA, shRNA, DNA vaccines, mucosal vaccines, modified viruses or viral components, and other bioactive molecules for gene therapy, wound healing therapies, cancer therapy, immune modulation, and use in research that requires delivery of DNA, RNA and/or proteins into mammalian cells and tissues.

Applications for Prevention of HIV/HSV

HIV is among the leading causes of global morbidity and mortality, currently infecting an estimated 40 million people and killing roughly three million people every year. Most HIV infections occur at one of two mucosal sites: either the genital-rectal mucosa via sexual contact or the oral-gastrointestinal mucosa via breastfeeding. Most adults acquire HIV through unprotected sex, while infants may acquire the virus from infected mothers during pregnancy, birth or breastfeeding. Condoms are effective in blocking sexual transmission of HIV, but are often not used. Abstinence and circumcision are only partially effective. Therefore, blocking HIV entry at the mucosal site is critical to prevent infection. The ultimate control of HIV transmission will rely on an effective, safe and inexpensive vaccine.

However, despite a global effort for more than two decades, an HIV vaccine remains elusive (Simon et al., Lancet 368:489-504, 2006). The human immune system is the target of HIV. Once infection establishes, the immune system breaks down. Unlike infections by other viruses, no natural recovery could be achieved after HIV infection. Current mucosal vaccines are either ineffective or unsafe, and vaccines given by injections do not induce adequate immunity in the mucosal tissues. Major obstacles include the genetic diversity and rapid mutation of the virus, the complex infection process, the immune system breakdown, the lack of an adequate animal model for testing vaccines, and the difficulty of designing an effective immunogen. HIV proviral DNA is integrated into the host genome soon after infection, so a vaccine must block HIV infection within hours of viral exposure at the port of viral entry. An ideal HIV vaccine should confer sterilizing immunity by eliciting broadly cross-reactive neutralizing antibodies (Abs) that block viral entry and aid in clearance of the infection. To date, most HIV vaccines are developed by priming cellular immune responses. These include induction of cross-reactive cytotoxic T lymphocytes (CTLs) that can kill virus-infected cells. Although such T-cell-based vaccines can control virus replication, they cannot prevent viral entry (Singh et al., Virol J. 3:60, 2006). To date, none of the current HIV vaccines can induce broadly cross-reactive neutralizing antibodies to block viral entry and provide ‘sterilizing immunity’ (Singh et al., 2006).

Prevention of HIV/HSV Infections by RNAi

Because developing an HIV vaccine has been challenging, an alternative preventative strategy may be to tackle its major accomplice—HSV-2. HSV-2 infection is a major cofactor of HIV transmission (Abu-Raddad et al., PLoS ONE 3(5) PLoS ONE 3(5): e2230, 2008), multiplying the risk of HIV acquisition up to five times (Freeman et al., AIDS 20:73-83, 2006; Kapiga et al., J Infect Dis 195(9):1260-9, 2007), presumably because of frequent ulcerations and the associated influx of activated CD4⁺ T cells that provide HIV easier access to high numbers of potential target cells (Koelle et al., J Infect Dis 169(5):956-61, 1994; Koelle et al., J Virol 68(5):2803-10, 1994). Co-infection with HSV-2 increases HIV transmissibility and accelerates the course of HIV disease. Further, HSV-2 shedding facilitates HIV shedding on mucosal surfaces. The two viruses interact synergistically (Heng et al., Lancet 343(8892):255-8, 1994). As a result, HIV infection causes more frequent HSV reactivation than in HIV-negative individuals.

While epithelial cells are the major targets for HSV-2, dendritic cells (DCs), lymphocytes, and macrophages are also susceptible to HSV-2 infection (Kucera et al., AIDS Res Hum Retroviruses 6(5):641-7, 1990), which triggers the down-modulation of their immunostimulatory functions. Subclinical reactivation of HSV-2 is associated with microscopic lesions that bring about an accumulation of activated CD4+ and CD8+ T cells, as well as DCs, which are target cells for HIV infection. HSV-2-infected people lacking obvious lesions exhibit increased susceptibility to HIV. Subclinical reactivation of HSV-2 is associated with microscopic lesions that bring about an accumulation of activated CD4+ and CD8+ T cells, as well as DCs, which are target cells for HIV infection. Due to the obvious role of HSV-2 in the promotion of HIV infection, two large scale clinical trials were conducted in Africa for HIV prevention by controlling HSV with the herpes drug acyclovir (Celum et al., N Engl J Med 362(5):427-439, 2010; Watson-Jones et al., N Engl J Med 358(15):1560-1571, 2008). Unfortunately, neither trial showed reduction in HIV infection.

Another recent study (Zhu et al., Nat Med 15(8):886-92, 2009) found that after HSV-2 infection, HIV-1 receptor expressing cells are recruited to the herpes lesion and persist for months after the lesion heals. This might partially explain the ineffectiveness of the herpes drug acyclovir for reducing HIV infection. By in situ analysis of the cellular infiltrate from sequential biopsies of HSV-2 lesions, Zhu et al. reported that CD4⁺ and CD8⁺ T cells, and dendritic cells (DCs), persisted at sites of HSV-2 reactivation for months after healing. The CD4⁺ T cells that persisted were enriched for CCR5 expression. This finding indicates that HSV infection increases both the HIV-receptor expression and the local concentration of cells expressing HIV receptors, neither of which is reversible by acyclovir.

Thus, silencing the viral entry receptors by RNAi could potentially prevent HSV and HIV infections. Both animal and human studies support this concept: the HSV receptor HVEM and nectin-1 double knockout mice are immune to HSV-2 (Taylor et al., Cell Host Microbe 2(1):19-28, 2007) and people inherited with CCR5 mutations are immune to HIV (Quillent et al., Lancet 351(9095):14-8, 1998). However, silencing HIV receptors locally in mucosal tissue (e.g., vaginal mucosa, oral mucosa, gastric/intestinal mucosa, olfactory mucosa, etc.) is challenging because normally few HIV target cells, such as CD4⁺ T cells, are exposed and accessible to siRNA. These cells are located deep in the mucosal tissue and may become exposed due to injuries, ulcerative lesions and inflammations. HSV-2 infection is the major cause of genital ulceration. Unlike HIV receptors (except syndecans), HSV receptors are located on surface epithelial cells that are more accessible to topically delivered siRNA.

To date, numerous receptors have already been identified for HSV (nectin-1, HVEM and pilr-α) and HIV (CD4, CCR5, CXCR4, and syndecans); and DC-SIGN is a receptor for both viruses on DCs (de Jong et al., J Gen Virol 89(Pt 10):2398-409, 2008). Developing better prevention strategies will require improved understanding of the initial infection events, interactions between HSV-2 and HIV-1, and identification of specific HSV-2 and HIV-1 infection receptors and/or other targets for preventing infection. Because about 90% of HIV+ individuals and about 19-50% of adults worldwide are already infected with HSV, more sexually active adults are experiencing HSV reactive than active infections. However, receptors have not been studied as a target for reactivation control.

The present disclosure provides RNAi delivery systems and methods that are safer, longer-lasting, more cost-effective, and suitable for human use. This technology uses delivery vehicles prepared from food-grade/commensal bacteria to deliver TransKingdom RNA interference (tkRNAi) (Xiang et al., Nat Biotechnol 24(6):697-702, 2006) and/or bacteria-mediated RNA interference (bmRNAi) (Brummelkamp et al., Science 296(5567):550-3, 2002; Zhang et al., Cancer Res 67(12):5859-64, 2007). Unlike the existing tkRNAi and bmRNAi, our unique method is food-grade and non-invasive. The RNAi system can also be used to study the role of viral receptors, identify and select potential targets for viral prevention (e.g., key receptor(s) in HSV and/or HIV infection), and study the mechanisms of HSV and HIV co-infection.

In one aspect, the novel noninvasive bacterial molecule-delivery systems described herein can be used to interfere with viral receptor gene expression by delivering shRNA to eukaryotic cells. In some examples, the bacterial vehicles may be loaded with exogenous shRNA molecules and may subsequently deliver the shRNA molecules to target eukaryotic cells via uptake of the vehicles by the cells. In other examples, the bacterial vehicles produce the shRNA and deliver the shRNA into the eukaryotic cells via uptake of the vehicles by the cells. In some embodiments, the shRNA is produced in excessive amounts in the vehicles prior to uptake by the eukaryotic cells. One example of such an RNAi delivery system is Noninvasive TransKingdom RNA interference (NtkRNAi).

In another aspect, the novel noninvasive bacterial molecule-delivery systems described above can be used to interfere with viral receptor gene expression by delivering shRNA genes encoding shRNA to eukaryotic cells. The eukaryotic cells transcribe the delivered shRNA genes to produce shRNA. One example of such an RNAi delivery system is Noninvasive Bacteria-Mediated RNA interference (NbmRNAi).

In contrast to current TransKingdom RNA interference (tkRNAi) and/or bacteria-mediated RNA interference (bmRNAi) delivery systems, these delivery systems provide delivery of RNAi molecules to eukaryotic cells using vehicles prepared from food-grade and/or non-invasive bacteria. Our bacterial delivery system has achieved a transfection rate of about 100% for DNA vaccines using noninvasive, native lactococcal and streptococcal strains. This achievement indicates that invasion or infection is not required for delivery by bacterial vectors. These delivery systems can be used to treat a disease or condition in vivo, to study the role of viral receptors, to identify potential targets for viral prevention, and to develop novel treatments for viral infections and other conditions.

For example, Gram-positive bacterial revenants can be modified to deliver shRNA (or plasmids encoding shRNA) targeting HSV-2 and HIV-1 receptors to mucosa (e.g., to the intestinal mucosa). A similar RNAi approach could be used to suppress one or more different viral receptors in the mucosa to achieve cell immunity to one or more other virus(es) in a safer, more efficient, and more cost-effective manner than currently available RNAi delivery systems. Likewise, these systems and methods may be used to deliver siRNA/genes targeted to non-viral mRNA or genes within mammalian cells. Persons with skill in the art will readily appreciate that such systems and methods are applicable to any disease or condition for which RNAi may be used as a treatment or prophylaxis. Various RNAi methods used in combination with the BDVs (BDVs) described herein may also provide greater effective duration of treatment.

Prevention of HIV Infection with Mucosal Vaccines

Two potential sites of action for an HIV vaccine are viral entry and viral replication. An ideal HIV vaccine should confer sterilizing immunity by eliciting broadly cross-reactive neutralizing Abs that block viral entry and aid in clearance of the infection. To date, most HIV vaccines are developed by priming cellular immune responses. These include induction of cross-reactive cytotoxic T lymphocytes (CTLs) that can kill virus-infected cells. However, such T-cell-based vaccines cannot prevent viral entry as is required for the prevention of transmission at the mucosal site of viral entry.

Current HIV vaccine designs are insufficient. Current HIV vaccine designs include the following major types: (i) Subunit vaccines: Recombinant DNA technologies are used to create vaccines containing a part of HIV that will trigger an immune response in the recipient. (ii) Live vector vaccines: A live bacterium or virus is used to express HIV proteins or deliver a gene that trigger an immune response. (iii) Peptide vaccines: Small pieces of HIV protein with ability to elicit strong immune responses. (iv) DNA vaccines: Viral DNAs rather than proteins are used to promote an immune response. (v) Pseudovirions: Synthetic viruses are used to inject genetic material, including DNA vaccines, into eukaryotic cells. Pseudoviruses are closely related to viruses in structure and behavior but cannot infect and replicate like true viruses. (vi) Virus-like particles (VLPs): Viral structural proteins form particles resemble the virus from which they were derived but lack viral nucleic acid, meaning that they are not infectious. VLPs used as vaccines are often very effective at eliciting both T cell and B cell immune responses. The human papillomavirus and Hepatitis B vaccines are the first virus-like particle based vaccines approved by the FDA.

Studying people who are naturally resistant to HIV infections (exposed seronegatives, or ESNs) may provide clues for vaccine development. Although ESNs include males, the most well-studied ESNs are female sex workers in Kenya and Thailand. The immune responses—both cellular and antibody—to HIV were found to exist in the cervix of these women (Belec et al., J Infect Dis 184:1412-1422, 2001; Beyrer et al., J Infect Dis 179:59-67, 1999; Broliden et al., Immunol Lett 79(1-2):29-36, 2001; Devito et al., AIDS 14:1917-1920, 2000; Devito et al., J. Acquir. Immune Defic. Syndr. 30:413-420, 2002; Ghys et al., AIDS 14:2603-2608, 2000; Kaul et al., J. Immunol 164:1602-1611, 2000; Hirbod et al., J Intern Med 262:44-58, 2007). Also, animal studies showed that when monkeys were exposed to subinfective doses of SIV, the “exposed uninfected” animals were protected from subsequent challenge with infectious SIV (Clerici et al., AIDS 8:1391-1395, 1994). The immunity in the female ESNs is likely induced by repeated vaginal exposures to subinfective HIV viruses (Cranage et al., Mucosal Immunol 3:57-68, 2009; Stamatatos et al., Nat Med 15:866-870, 2009; Laurence, J Reprod Immunol 58(1):79-91, 2003). This indicates that live viruses can induce sterilizing immunity, but HIV is too dangerous to be developed as a live vaccine for human use.

To date, the best HIV vaccine reported is an injected canarypox vector vaccine as prime plus a recombinant gp120 subunit vaccine as boost, which achieved only a modest 31% protection (Rerks-Ngarm et al., N Engl J Med 361:2209-2220, 2009). Apparently, new designs are needed to develop more effective HIV vaccines.

Most HIV vaccines currently in clinical trials are given by injection. This route can produce strong immune response in the blood, but not at the mucosal surfaces (Neutra et al., Nat Rev Immunol 6:148-158, 2006).

Nasal vaccines show promise but are unsafe. The human immune system consists of two compartments: the systemic (serum) and mucosal. Although transfusion and contaminated needles can transmit HIV, most HIV infections occur at mucosal sites via sexual contact or breastfeeding. The route of vaccine administration affects the level of the immune response in the systemic and mucosal tissues. Mucosally-administered vaccines, including those by the oral, nasal, rectal or vaginal route, are more effective at producing responses in mucosal tissues (Xin et al., Blood 102:223-228, 2003). In rodents vaccines administered nasally can induce potent immunity in the respiratory tissues and in the vagina (Mestecky et al., Am J Reprod Immunol 53:208-214, 2005; Staats et al., AIDS Res Hum Retroviruses 13:945-952, 1997; Vajdy et al., Expert Opin Drug Deliv 3:247-59, 2006). These studies show promise that nasal vaccination may prevent sexual transmission of viruses in the females. However, animal studies have shown that brain infections occurred after nasal immunization of a live adenovirus-based HIV vaccine (Lemiale et al., J Virol 77:10078-10087, 2003). Because non-viable vaccines often induce weak responses to HIV, live-vectors may be needed (Spearman et al., Curr Pharm Des 12:1147-1167, 2006). For safety reasons, a mucosal vaccination not using the nasal passage is preferred.

Various bacteria have been used to deliver HIV vaccines to the mucosa. The commensal, non-invasive bacteria-based vaccines can induce mucosal immunity, but their potency is often weak (Lee, Curr Opi Infect Dis 16:231-235, 2003). Invasive bacteria including Listeria, Salmonella, Shigella, and Yersinia or food bacteria expressing a cloned invasion gene have been used to deliver oral vaccines to the gut (Guimarães et al., 2006; 2009). These bacteria-based vaccines are not safe for human use.

As further described below, we have made a technological breakthrough that enables non-invasive food/commensal bacteria to deliver vaccines, RNAi molecules, and other cargo safely and efficiently to the mucosa and to other targeted mammalian tissues. In some examples, the bacterial delivery vehicles (BDVs) transfect eukaryotic cells of a target tissue with at least 90% efficiency, at least 95% efficiency, or 100% efficiency (i.e., all cells of the target tissue are transfected).

### Example 1

### Novel Plasmids: Shuttle Plasmid Vectors, Protein Reporter Plasmids, and DNA Vaccine Reporter Plasmids

Existing gene-delivery plasmids, such as pcDNA3.1 (Invitrogen), are designed for E. coli and cannot replicate in Gram-positive bacteria. We designed and constructed a novel E. coli-LAB shuttle plasmid vector, pLKV1, for the delivery of DNA into eukaryotic cells. This plasmid has several advantages over many existing plasmids for DNA vaccine delivery, such as those described by Gram et al. (Genet Vaccines Ther 5:3, 2007) and Guimarães et al. (2005; 2006; 2009). First, it can be expressed in both Gram-negative (e.g., E. coli) and Gram-positive bacteria (e.g., LAB species, including Lactococcus, Lactobacillus, Streptococcus and Weisseilla). This feature allows relatively easy construction of DNA vaccines in E. coli and subsequent transformation into a suitable strain (e.g., a LAB strain) for vaccine delivery. Second, it has a small size (4.4 kb), so it can be used to clone DNA vaccines of large sizes, which can be easily transformed into various bacterial species. Large plasmids are often difficult for gene cloning and transformation. Third, it has a high copy number (about 65 per cell in LAB and comparable to pUC18 in E. coli), so that it can deliver DNA vaccines with a higher dose. Fourth, it carries a kanamycin resistance marker that expresses in both Gram-negative and Gram-positive bacteria. This feature is desirable because kanamycin resistance marker is recommended by the FDA for use in DNA vaccine construction due to a better safety record than other antibiotic resistance markers (FDA, 1996).

To monitor gene delivery into mammalian cells, the red fluorescent protein (Rfp) gene (DsRed2, Clontech) was inserted to form pLKV-Red2. The pLKV-Red2 plasmid was tested in several BDVs for delivery into Caco-2 cells (see e.g., Examples 2 and 3).

pLKV1 Vector Design and Construction:

pLKV1 contains three parts—a plasmid replication origin of pA1 derived from Lactobacillus plantarum (Vujcic et al., Appl Environ Microbiol 59:274-280, 1993), which replicates in both Gram-negative and positive bacteria; a kanamycin resistance marker derived from Tn1545 of Streptococcus pneumoniae (Courvalin et al., Mol Gen Genet 206:259-264, 1987) and can express in both Gram-negative and positive bacteria; and an eukaryotic gene expression cassette derived from pVAX1 (Invitrogen, Carlsbad, Calif.). Due to safety records, kanamycin resistance marker is recommended for DNA vaccine delivery by the Food and Drug Administration (FDA). Therefore, the construction of the vaccine delivery vector included two steps.

First, a small-sized, 3.7 kb kanamycin resistant E. coli-LAB shuttle plasmid pLK1 was constructed by combining a 2.1 kb DNA fragment containing the pA1 origin from the plasmid pA13 and the 1.6 kb DNA fragment containing the kanamycin resistance marker of Tn1545 from pAK267.

Second, an 860-bp fragment containing a DNA vaccine cloning cassette, a CMV promoter, a multiple cloning site, and a poly-A tail, was obtained from pVAX1. The DNA fragment was generated by polymerase chain reaction (PCR) with TaKaRa e2TAK™ DNA Polymerase (Fisher Scientific) and primers pVAX1-F (5′ GGAGATCTGCGTTACATAACTTACGG 3′; SEQ ID NO: 1) and pVAX1-R (5′ TAGAAGCCATAGAGCCC 3′; SEQ ID NO: 2) introducing respectively a BglII (underlined) and a blunt site in the fragment. PCR was performed using the Techne thermalcycler (Techne, Princeton, N.J., USA). The reaction mixture (final volume of 50 μl) contained: 100 ng template DNA; 1 unit e2TAK™ DNA polymerase; 1× reaction buffer; deoxynucleoside triphosphates 0.1 mmol I⁻¹ each; primer, 50 pmol each. The thermal cycling program used was as follows: initial denaturation at 94° C. for 3 min, and 35 cycles of 94° C. for 45 s, 55° C. for 1 min, and 72° C. for 1 min. Finally, there was an extension step at 72° C. for 10 min. The PCR DNA products were analyzed for correct size and purity on 1.2% agarose gel.

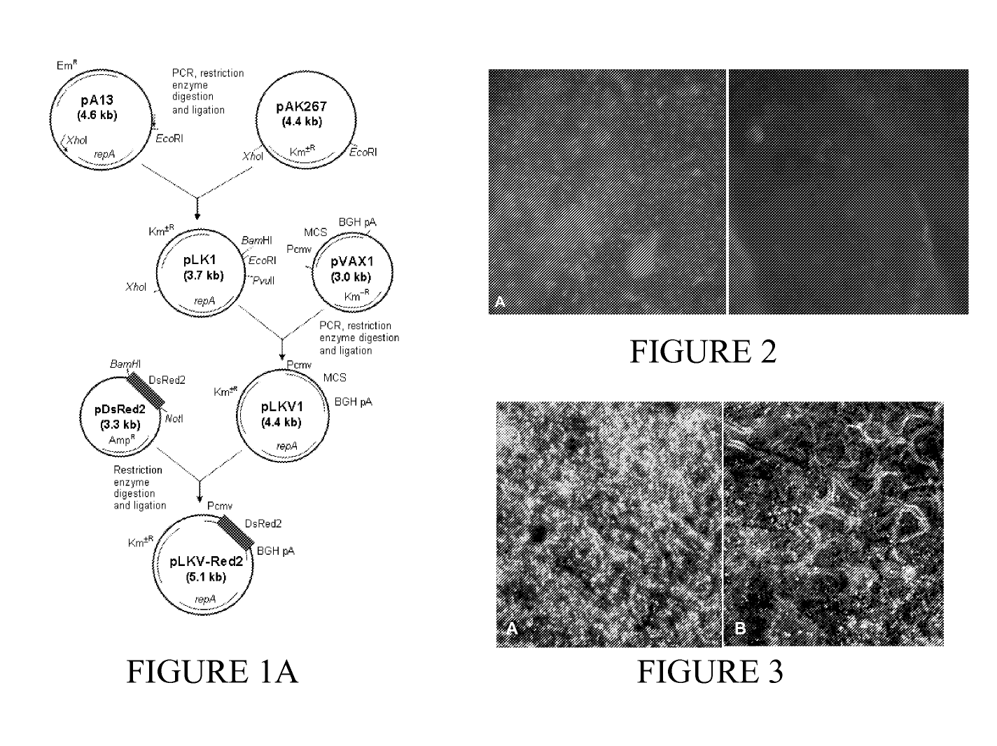

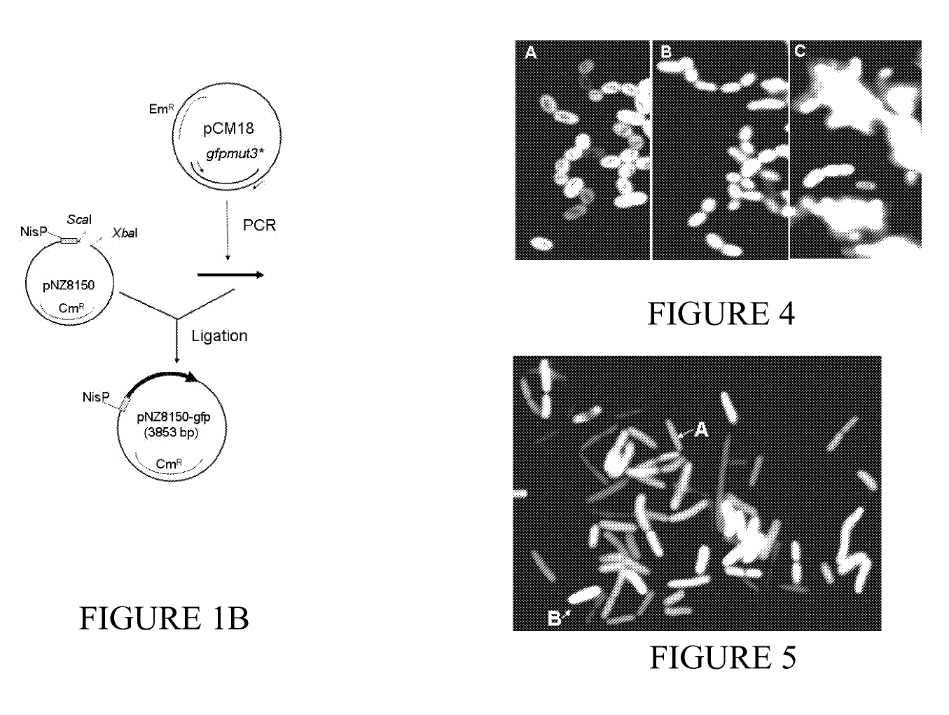

The amplified PCR product was cut with BglII and cloned into the shuttle vector pLK1 predigested with BamHI and PvuII (FIG. 1) resulting in pLKV1. Due to a 0.3-kb deletion in pLK1 after the double enzyme digestion, the size of pLKV1 was 4.4 kb. The plasmid was established by transformation in E. coli DH5α and then in L. lactis NZ3900 and S. gordonii strains (Table 1). Automatic sequencing was used to confirm the integrity of the pLKV1 sequence and to determine the size and nucleic acid sequence of pLKV1. The size was determined to be 4,448 bp. The sequence was deposited in GenBank with an accession number of HM569775 (SEQ ID NO: 8).

pLKV1 Characteristics:

As shown in FIG. 1, pLKV1 contains the eukaryotic region with the CytoMegaloVirus promoter (pCMV), a multiple cloning site (MCS), and the polyadenylation signal of bovine growth hormone (BGH polyA) needed for a gene expression by eukaryotic host cells. Its prokaryotic region contains the RepA replication origin for a broad host range expression in both E. coli and LAB strains. The MCS inserted between the eukaryotic promoter pCMV and the BGH polyA, provides five unique restriction enzyme recognition sites, BamHI, SpeI, EcoRI, PstI and NotI, which can be used to clone a gene of interest and the T7 primer binding site for its sequencing.

Reporter Plasmid Construction:

To facilitate vaccine development, we constructed novel protein and DNA delivery reporter plasmids with the green and red fluorescent protein genes, respectively. These reporter vaccine plasmids not only can be used for monitoring in vitro DNA uptake by cultured cells, but also can be used for monitoring DNA uptake in vivo in animals. The uptake and expression of these fluorescent reporter vaccines in a small animal can be monitored in real time with a noninvasive whole body imaging system, such as the UVP iBox® Scientia Small Animal Imaging System (www.uvp.com/ibox.html).

DNA Delivery Reporter Plasmid:

A promoterless 0.7-kb rfp ('rfp) cassette encoding the red fluorescent protein DsRed2 was obtained from the plasmid pDsRed2 (Clontech) by digestion with restriction enzymes BamHI and EcoRI. It was subcloned into pLKV1 multiple cloning site (MCS) downstream of the CMV promoter resulting in pLKV-Red2 (5.1 kb). The new plasmid carries a reporter DsRed2 gene that expresses only in eukaryotic cells. This plasmid was used to evaluate the delivery and functionality of the DNA vaccine plasmid pLKV1 (i.e., plasmid pLKV1 with a DNA vaccine sequence inserted into the MCS).

Protein Expression Reporter Plasmid:

A promoterless 0.72-kb gfp ('gfp) cassette encoding the GFPmut3* was amplified from pCM18 (Hansen et al., Microbiology 147(Pt 5):1383-1391, 2001) by PCR with the TaKaRa e2TAK™ DNA Polymerase. The primers were Gfpm3-F1 (5′-atgcgtaaaggagaaga actt-3′ SEQ ID NO: 3) and Gfpm3-Rx (5′-tagctctagattattatttgtatagttcatcc-3′; SEQ ID NO: 4) introducing one blunt end and one sticky end (XbaI, underlined). The PCR condition was the same as above. The DNA fragment was subcloned into the ScaI and XbaI site of pNZ8150 to yield pNZ8150-gfp (3,853 bp)(FIG. 1B, pNZ8150-gfp; SEQ ID NO: 9). The 'gfp cassette encodes the mutant GFP protein GFPmut3*, which exhibits 100-fold-greater fluorescence intensity than the wild-type GFP protein when expressed in E. coli (Cormack et al., Gene 173:33-38, 1996). The 'gfp cassette in pNZ8150-gfp expresses the GFPmut3* protein only in bacteria containing the nisin-controlled gene expression system (NICE)(Mierau et al., Appl Microbiol Biotechnol 68:705-717, 2005). The fluorescent protein expresses at such a high level that the bacteria show bright green color and can be visible after entering the eukaryotic cells. The expression of the green fluorescent protein in L. lactis NZ3900 cells was confirmed under an epifluorescent microscope (Olympus Model BX50F) after nisin induction suggested by the supplier (MoBiTec).

In summary, a delivery shuttle vector may include a promoter upstream of a cloning site and a first antibiotic resistance marker that is expressible in both Gram-positive and Gram-negative bacteria. A DNA delivery reporter plasmid may include a desired DNA sequence, such as a cassette encoding a first reporter protein (e.g., DsRed2), inserted into the MCS of the delivery shuttle vector. The first reporter protein may be expressible in eukaryotic cells and not expressible in bacteria. A protein expression reporter plasmid may be a nisin-induced expression vector that includes a DNA sequence encoding a second reporter protein, and the second reporter protein may be expressible in both Gram-positive and Gram-negative bacteria (e.g., GFPmut3*). The protein expression reporter plasmid may also include a second antibiotic resistance marker that is expressible in both Gram-positive and Gram-negative bacteria.

A DNA sequence encoding a DNA vaccine or RNAi molecule can be inserted into the MCS of the delivery shuttle vector, and the delivery shuttle vector may be inserted into a BDV for delivery to a eukaryotic cell. The DNA delivery reporter plasmid and/or protein expression reporter plasmid may be inserted into the same BDV or into another BDV to confirm delivery of the DNA and/or protein expression, respectively, in the eukaryotic cell. Thus, these novel plasmids provide a mechanism for delivering a cargo to a eukaryotic cell and for monitoring the efficiency of delivery, transfection, and/or expression in the eukaryotic cell, as described in further detail in the following Examples.

### Example 2

### Delivery of DNA to Eukaryotic Cells by Gram-Positive BDVs

In this Example, cell wall-stripped bacterial delivery vehicles (BDVs) were used to deliver the plasmids described above in Example 1 to eukaryotic cells. Stripping the bacterial cell walls resulted in increased internalization of BDVs and their cargo (plasmids) by the target cells. These results demonstrate that BDVs provide highly efficient delivery of cargo to eukaryotic cells.

Bacterial Strains, Plasmids, Media, and Growth Conditions:

Bacterial strains and plasmids used in this study are listed in Table 1. Lactococcus lactis subsp. cremoris NZ3900 was grown in M17 medium containing 0.5% glucose (GM17) and Streptococcus gordonii V288 was grown in Todd Hewitt broth (Difco Laboratories). Escherichia coli strains were grown on Luria-Bertani medium and incubated at 37° C. with vigorous shaking. Antibiotics were added at the indicated concentrations as necessary: kanamycin, 600 μg/ml for L. lactis; 250 μg/ml for S. gordonii, 50 μg/ml for E. coli.

TABLE 1
Bacterial strains and plasmids

| | | Source/ |
|---|---|---|
| Strain/plasmid | Characteristics* | Reference |
| E. coli DH5α | (F-80dlacZΔM15 Δ(lacZYA- argF) | Invitrogen |
| | U169 endA1 recA1 hsdR17 | |
| | (rk− mk+) deoR thi-1 supE44 | |
| | λ- gyrA96 relA1) | |
| L. lactis NZ3900 | lacF−, pepN::nisRnisK; food-grade | MoBiTec |
| | expression strain | |
| S. gordonii V288 | Oral commensal strain | F. Macrina |
| NZ3900-pLKV- | NZ3900 harboring pLKV-Red2/ | This study |
| Red2 | Km^{±R} | |
| NZ3900- | NZ3900 harboring pNZ8150-gfp/ | This study |
| pNZ8150-gfp | Cm^{R} | |
| V288-pLKV- | V288 harboring pLKV-Red2/ | This study |
| Red2 | Km^{±R} | |
| pA13 | E. coli-LAB shuttle cloning | L. Topisirovic |
| | plasmid/Em^{R} | |
| pAK267 | Plasmid carrying the Km^{±R} cassette | This study |
| | from Tn1545 | |
| pVAX1 | Expression vector containing | Invitrogen |
| | pCMV, MCS and BGH polyA/ | |
| | Km^{-R} | |
| pCM18 | Plasmid carrying the gfpmut3* | Hansen et al, |
| | gene/Em^{R} | 2001 |
| pDsRed2 | Plasmid carrying the DsRed2 gene/ | Clontech |
| | Amp^{R} | |
| pNZ8150 | Nisin-induced expression vector/ | MoBiTec |
| | Cm^{R} | |
| pNZ8150-gfp | pNZ8150 containingthe gfpmut3* | This study |
| | gene/Cm^{R} | |
| pLK1 | E. coli-LAB shuttle cloning | This study |
| | plasmid/Km^{±R} | |
| pLKV1 | E. coli-LAB shuttle DNA vaccine | This study |
| | plasmid/Km^{±R} | |
| pLKV-Red2 | Red fluorescent reporter DNA | This study |
| | vaccine plasmid/Km^{±R} | |

*Amp^{R}: ampicillin resistance; Cm^{R}: chloramphenicol resistance; Em^{R}: erythromycin resistance; Km^{-R}: kanamycin resistance only in Gram-negative bacteria; Km^{±R}: kanamycin resistance in both Gram-negative and positive bacteria.

Genetic Transformation of S. gordonii V288:

A natural transformation protocol was used to transform S. gordonii V288 with the pLKV-Red2 plasmid, in which the red fluorescent protein gene is expressed only in eukaryotic cells but not in prokaryotic cells. Briefly, V288 was grown for 16 h in Todd-Hewitt broth supplemented with 5% heat-inactivated horse serum at 37° C. The culture was diluted 1:40 in the same broth and continued to incubate for exactly 2 h. 0.1 μg of the plasmid pLKV-Red2 DNA was added to the culture. After 1 h, the bacterial cells were plated on Todd-Hewitt agar supplemented with kanamycin at 250 μg. Transformants were isolated after 24 h incubation in a candle jar.

Genetic Transformation of L. lactis NZ3900:

A lactococcal culture was grown in 5 ml GM17 broth overnight at 30° C. The culture was then inoculated into 20 ml pre-warmed GM17 broth and incubated at 30° C. for 2-3 h to reach early exponential phase (OD₆₀₀=0.3-0.6). Penicillin G was added to a final concentration of 100 μg/ml and the culture was incubated for 1 h. The cells were harvested by centrifugation and resuspended in 1 ml lithium acetate solution (100 mM LiAc; 10 mM DTT; 0.6 M sucrose; 10 mM Tris-HCl, pH 7.5; filter-sterilized) and incubated for 30 min at room temperature. The cells were washed with sterile deionized water twice, 50 mM EDTA once, and sterile deionized water three times, and finally resuspended in 2 ml 0.3 M sucrose. The resuspended cells could be stored at −80° C. for future use, or used directly in the electroporation protocol described below.

The plasmid pLKV-Red2 DNA was added at 0.1 μg into 80 μl cells in a 1 mm pre-cooled electroporation cuvette. The Bio-Rad Gene Pulser II Electroporation system was used. The voltage was set at 1.8 kV, resistance at 400Ω, and capacitance at 25 μF. After delivery of the electric pulse, 1 ml recovery medium (GM17 supplemented with 0.3 M sucrose) was added and the cells were retrieved into an eppendorf test tube and incubated at 30° C. for 2 h. The cells were plated on GM17 agar supplemented with kanamycin at 600 μg/ml or chloramphenicol at 10 μg/ml for selection of pLKV-Red2 or pNZ8149-gfp, and incubated in a candle jar for 24-48 h at 30° C.

Transfection Assays of pLKV-Red2 into Caco-2 Cells

The pLKV-Red2 plasmid was assayed for DsRed2 expression in the human colon cancer cells (Caco-2 cells). Fifty to 80% confluent Caco-2 cells were cultured in Dulbecco modified Eagle medium, 10% fetal calf serum, 2 mM L-glutamine (BioWhittaker, Cambrex Bio Science, Verviers, Belgium), 100 U penicillin and 100 g streptomycin.

Caco-2 cells were transfected with 1.0 μg of pLKV-Red2 or pLKV1 (negative control) with Lipofectamine™ reagent (Invitrogen). The red fluorescent protein-producing cells were visualized 48 h after transfection with an epifluorescent microscope (Zeiss, Axio Observer Z1 Motorized Inverted Research Microscope). We observed red fluorescent protein (RFP) expression in these epithelial cells at 48 h after transfection, but no RFP expression was observed after transfection with the blank shuttle DNA-delivery vector pLKV1 (data not shown). This result demonstrated that pLKV1 is functional for gene expression in mammalian cells.

Gene Transfer from Bacteria to Caco-2 Cells:

To demonstrate the efficacy of pLKV1 as a delivery vector with noninvasive food and commensal bacteria, both L. lactis NZ3900 and S. gordonii V288 containing pLKV-Red2 were used for gene delivery into Caco-2 cells. The bacteria were treated with glycine, penicillin, lysozyme and/or electroporation to weaken the cell walls in order to facilitate the uptake of the bacteria by the Caco-2 cells.

L. lactis NZ3900 and S. gordonii V288 were transformed with pLKV-Red2, and L. lactis NZ3900 was transformed with pNZ8150-gfp. To facilitate the gene transfer, L. lactis NZ3900 and S. gordonii containing pLKV-Red2 and L. lactis NZ3900 containing pNZ8150-gfp were treated with several different methods to weaken the cell wall prior to their addition to the mammalian cells.

The L. lactis strain was grown at 30° C. in GM17 supplemented with 600 μg/ml kanamycin for selecting pLKV-Red2 or 10 μg/ml chloramphenicol for selecting pNZ8150-gfp. The S. gordonii strain was grown at 37° C. in Todd-Hewitt broth supplemented with 200 μg/ml kanamycin.

For penicillin or glycine treatment, overnight cultures were diluted 1:5 (2 ml into 8 ml) with pre-warmed medium and grown for 2 h. Penicillin (100 μg/ml) or glycine (2.5% w/v) was added, after which the cultures were continued for 1 h and harvested. Harvested cultures were washed 3 times with sterile deionized water and resuspended in 0.5 ml 10% glycerol. At this stage, the cells were either electroporated or treated with lysozyme without electroporation.

For lysozyme treatment, the bacterial cells in 10% glycerol were treated with lysozyme at 20 μg/ml and incubated for 1 h at 37° C. The cells were then washed twice with 10% glycerol to remove the lysozyme and resuspended in 0.5 ml 10% glycerol.

For electroporation treatment, the Bio-Rad Gene Pulser II Electroporation system and 4 mm cuvettes were used. The voltage was set at 2.5 kV, resistance at 400Ω, and capacitance at 25 μF. After the delivery of electric pulse, the cells were kept on ice and used within 2 h.

Assays of bacteria into human cells were performed using the human colon carcinoma cell line Caco-2. Briefly, eukaryotic cells were cultured in P6 wells plates containing 1×10⁶ cells per dish in RPMI supplemented with 2 mM L-glutamine and 10% fetal calf serum. The multiplicity of infection (M01) was 10³ bacteria/cell. After 3 h of culture at 37° C. to allow internalization of bacteria, cells were washed 5 times to remove extracellular bacteria. The expression of fluorescent proteins in Caco-2 cells and in L. lactis bacteria were visualized with an epifluorescent microscope (Zeiss, Axio Observer Z1 Motorized Inverted Research Microscope).

FIGS. 2A-B and 3A-B illustrate the effect of cell wall treatments on gene transfer from bacteria to Caco-2 cells. In the absence of a bacterial cell wall treatment, the gene transfer by both bacteria was near zero (FIGS. 2B and 3B).

For delivery of the reporter gene by S. gordonii V288, treatment of the bacteria with penicillin and lysozyme gave the best result, with nearly all Caco-2 cells showing strong red fluorescence (FIG. 2A; delivery by untreated S. gordonii V288 without treatment with penicillin and lysozyme shown in FIG. 2B). Electroporation of the bacteria to weaken the cell wall did not further improve the transfection result.

For delivery of the reporter gene by L. lactis NZ3900, treatment of the bacteria with glycine gave the best result (i.e., strongest red fluorescence of Caco-2 cells; data not shown). Additional treatment with lysozyme and electroporation did not further improve this effect.

As shown in FIGS. 3A-B, improved gene transfer is associated with increased internalization of the bacteria into the mammalian cells. L. lactis NZ3900 bacteria containing pNZ8150-gfp, which turns the bacteria fluorescent green, were pretreated with 2.5% glycine (FIG. 3A; without glycine pretreatment, FIG. 3B). Glycine pretreatment of the bacteria significantly increased uptake of the bacteria by Caco-2 cells. These results demonstrate that improved internalization of these bacterial vectors and improved gene transfer can be achieved by pretreatment of the bacterial cell walls.

Pretreatment of the bacterial cell walls may include physically and/or chemically stripping the cell walls using various chemicals and/or physical methods. In other examples, the cell wall of a gram-positive bacterium may be chemically stripped by treating the bacterium with one or more of penicillin, vancomycin, a β-lactam antibiotic, a glycopeptide antibiotic, DL-threonine, glycine, nisin, phage lysin, and lysozyme. For example, bacterial cell walls can be weakened and partially stripped by addition of DL-threonine and/or glycine into the growth medium. Alternatively, the cell wall may be physically stripped by applying one or more of electroporation, heat, or mechanical force (e.g., mixing with glass beads) to the bacterium. As still another alternative, the cell wall may be physically stripped and chemically stripped using a combination of one or more of the above chemicals and methods.

### Example 3

### Delivery of Nucleic Acids by Gram-Positive Bacterial Revenants

In general, epithelial cells are difficult to transfect. A number of viral based strategies have been developed for gene delivery into the epithelial cells (Waehler et al., Nat Rev Gen 8:573-587, 2007). Nevertheless, these viral-based approaches can be time-consuming and costly.

A new strategy for delivering DNA to mammalian cells involves the use of bacterial ghosts to target antigen-presenting cells and other eukaryotic cells for gene delivery and vaccination. Bacterial ghosts are produced by protein E-mediated lysis of Gram-negative bacteria carrying the plasmid encoding the lysis gene E of bacteriophage Ph1×174 (Xin et al., 2003). The phage lysin forms transmembrane tunnels through the bacterial cell, resulting leakage of the cytoplasm content to form an empty bacterial cell envelope (Witte et al., Arch Microbiol 157:381-388, 1992). Bacterial ghosts retain all morphological and structural features of the cell wall and can be used as a delivery system for proteins, nucleic acids, and therapeutic drugs (Lubitz et al., Adv Exp Med Biol 655:159-170, 2009). Bacterial ghosts provide a novel non-viral tool for gene delivery to cells that are difficult to transfect, such as the UM1 oral epithelial cell lines used in this Example (below).

To date, only pathogenic Gram-negative bacteria, including Escherichia coli (Paukner et al., Mol Ther 11:215-233, 2005), Mannheimia haemolytica (Ebensen et al., J Immunol 172:6858-6865, 2004), and Vibrio cholerae (Ekong et al., FEMS Immunol Med Microbiol 55:280-291, 2009), have been used to produce bacterial ghosts, possibly due to host range limitation of the lysin protein E of bacteriophage PhiX174. Using pathogenic Gram-negative bacteria to produce bacterial ghosts raises safety concerns, because lipopolysaccharide on the envelopes of Gram-negative bacteria has endotoxin activity even after the bacteria are dead (Mader et al., Vaccine 15:195-202, 1997). This potential safety hazard may limit the clinical utility of pathogenic Gram-negative bacterial ghosts. Recently, a new method using cell membrane penetrating peptides has been developed to produce bacterial ghosts with E. coli (Palm-Apergi et al., J Control Release 132:49-54, 2008).

Conventional pathogenic Gram-negative bacterial ghosts deliver genes with relatively high efficiency into macrophages and dendritic cells (Haslberger et al., J Biotechnol 83:57, 2000; Paukner et al., J Drug Target 11:151-161, Staats et al., 1997; Vajdy et al., 2006). This is not surprising because these cells are professional phagocytes that can engulf dead bacteria by phagocytosis. In contrast, the E. coli-derived bacterial ghosts deliver genes into non-professional phagocytes (e.g., epithelial cells, such as Caco-2 cells) with relatively low efficiency (Vujcic et al., 1993). Thus, conventional bacterial ghosts may have limited use for the delivery of genes into cells other than professional phagocytes.

In addition, most or all of the internal contents of the original bacterium are removed (i.e., through bacterial lysis and washing/purification) during the preparation of the bacterial ghosts. Thus, conventional bacterial ghosts are essentially broken envelopes of bacterial cell wall. Using conventional bacterial ghosts for delivery of genes to target cells requires production of the genes and bacterial ghosts in separate processes. The bacterial ghosts must then be loaded with the genes in an additional process.

BDVs described herein can be produced by simpler and more efficient methods than bacterial ghosts. This Example describes the transfection of eukaryotic cells by novel bacterial revenants, a type of BDV prepared by perforating or puncturing the plasma membrane of a wall-stripped non-invasive bacterium. The plasma membrane may be perforated by treating the wall-stripped non-invasive bacterium with nisin, which forms pores or tunnels through the bacterial plasma membrane. (Alternatively, as demonstrated in Example 2, the plasma membrane may be perforated by treating the wall-stripped non-invasive bacterium with lysozyme.)

The perforation of the plasma membrane may cause the expulsion of some, but not all, of the intracellular contents of the bacteria. Thus, unlike a “bacterial ghost,” which retains very little or none of the internal contents of the bacterium, a “bacterial revenant” is still largely intact and retains some or all of its internal cargo or contents (particularly larger molecules and plasmids) after perforation of the plasma membrane. Therefore, a bacterium can be used to produce a cargo (e.g., produce a protein or replicate a plasmid) and subsequently converted into a loaded BDV by stripping the cell wall (and optionally, perforating the plasma membrane).

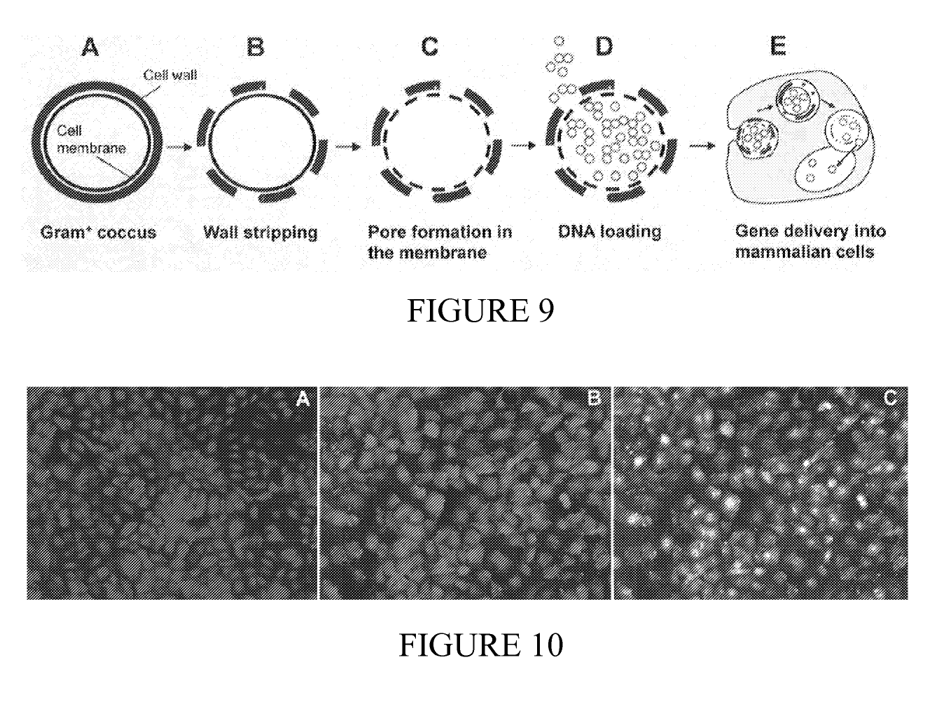

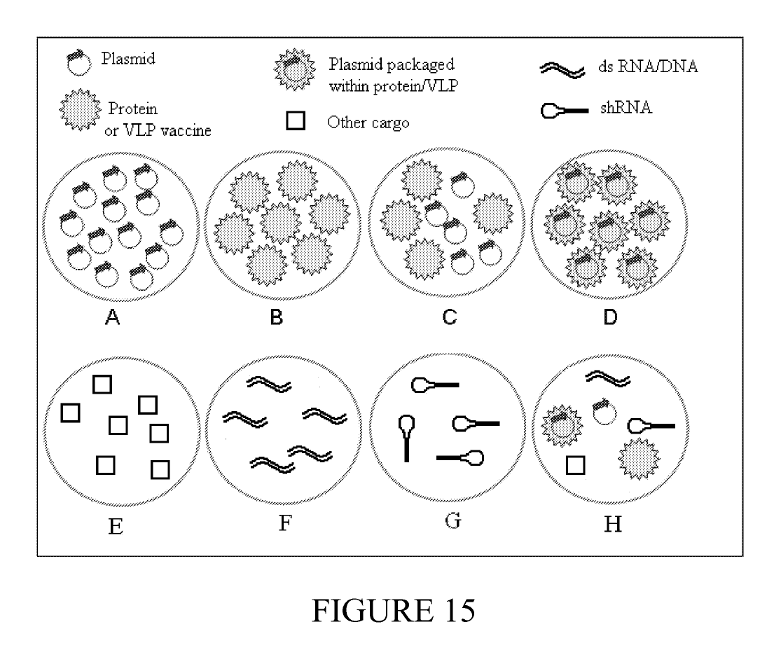

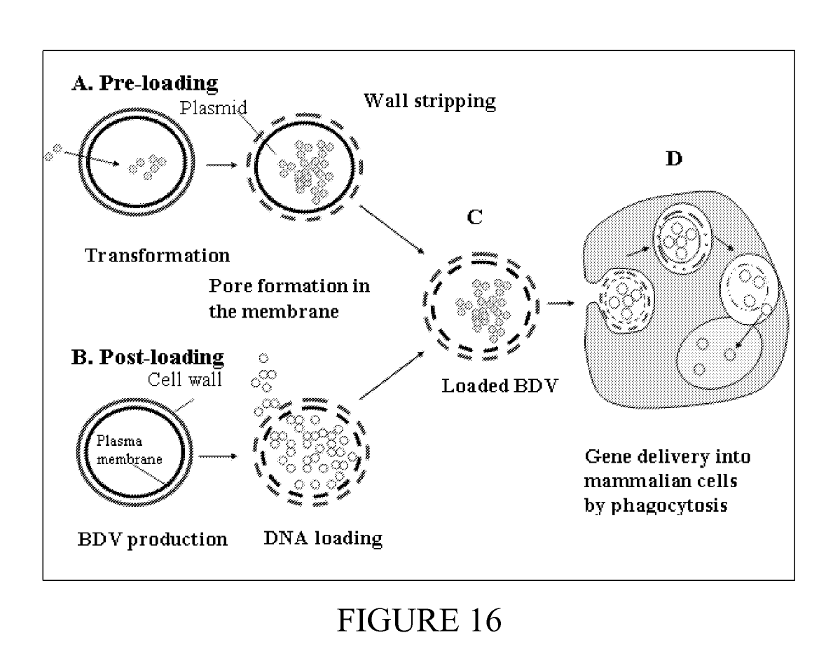

Optionally, BDVs or bacterial revenants may be heated for 5 minutes at 65° C. to inactivate any remaining live bacteria. In some examples, the BDVs or bacterial revenants may be heated after treating the bacteria with one or more chemicals to strip the cell walls. In other examples, bacterial revenants may be heated after treating the bacteria with one or more chemicals to perforate the plasma membrane (e.g., lysozyme, nisin, or an inducer of a gene in the bacterium that results in production of such a chemical). The bacterial revenants may be loaded with additional cargo after the cell membrane is perforated (see FIG. 9D; see also FIGS. 15 and 16).

As shown in FIGS. 15 and 16, BDVs and bacterial revenants provide several options for delivery of cargo to eukaryotic cells. BDVs can be loaded with any one or more of a plasmid, a vaccine, a protein, nucleic acids, and other cargo (e.g., a nanoparticle, a drug, etc.; FIG. 15). The bacteria used to prepare the bacterial revenants can be loaded with cargo (pre-loading) and the plasma membrane may subsequently be punctured, with at least some of the cargo remaining within the bacterial revenant for delivery. The bacteria used to prepare the bacterial revenants can be transformed with one or more nucleic acids for production of a cargo (pre-loading), the bacteria may produce the cargo, and the plasma membrane may be subsequently punctured, leaving some of the produced cargo within the bacterial revenant for delivery. After perforation of the plasma membrane, a bacterial revenant can be loaded with exogenous cargo (post-loading) such as synthetic nucleic acids or drugs. Two or more of these methods may be used in any combination (e.g., pre-loading and post-loading different cargo into one delivery vehicle; see also FIG. 16).

In this Example, the bacterial revenants are prepared from food-grade/commensal Gram-positive bacteria. However, bacterial revenants may be prepared from other non-invasive Gram-positive or Gram-negative bacteria in accordance with embodiments of the present disclosure.

As demonstrated below, the Gram-positive bacterial revenants of this Example deliver reporter genes encoding red and green fluorescent proteins into oral epithelial cells with high transfection efficiencies. In addition to UM1 cells, it was tested equally effective with Caco-2 cells (data not shown). Unexpectedly, our results demonstrate that the uptake of loaded Gram-positive revenants by eukaryotic cells does not require bacterial invasion or the use of virulence genes for delivery. Dead bacteria do not invade; therefore, invasion is absolutely not required for efficient gene delivery into the mucosal cells. This indicates that the human mucosal epithelial cells might act as “nonprofessional” phagocytes that can take up the bacterial revenants.

These data indicate that we can deliver any molecules, including RNAi, into mucosal cells with a noninvasive bacteria-based system. The mechanism for BDV internalization by the epithelial cells is unknown. We propose several possibilities. One, live and intact bacteria may be more resistant to phagocytosis by “non-professional” phagocytes, such as the mammalian epithelial cells, but dead and cell wall damaged bacteria may be more susceptible to such “non-professional” phagocytosis. Two, the surface of food or commensal bacteria may carry specific markers recognizable by the mammalian gut epithelial cells (Artis, Nat Rev Immunol 8:411-420, 2008). Disrupting the bacterial cell wall may render these markers unrecognizable. Three, the bacteria may carry phagocytosis-associated receptors embedded in the cell wall or its associated structures (Melendez et al., 2008). Disrupting bacterial cell wall could expose such receptors and subsequently result in phagocytosis by mammalian epithelial cells.

One method for the production and use of a Gram-positive bacterial revenant is illustrated in FIGS. 9A-E. FIG. 9A illustrates a Gram-positive coccus with a thick cell wall. As shown in FIG. 9B, the bacterial cell wall is weakened or partially stripped by glycine, penicillin and/or lysozyme. The bacterial cell membrane is perforated by one or more chemicals (e.g., nisin or lysozyme) to form a bacterial revenant (FIG. 9C). A cargo, such as plasmid DNA, is loaded into the bacterial revenant (FIG. 9D). The loaded bacterial revenant is added to a eukaryotic cell, which engulfs the loaded bacterial revenant by endocytosis. The bacterial revenant is degraded in the eukaryotic cell (e.g., within a vacuole, endosome, phagosome, or lysosome), releasing the cargo. The cargo may be delivered to a particular location within the eukaryotic cell (e.g., to the nucleus; FIG. 9E).

Expression System and Reporter:

A green fluorescence protein (Gfp)-tagged bacterium was used to monitor bacteria uptake by mucosal cells. Because Gfp expresses poorly in Gram-positive bacteria, a food-grade nisin controlled expression (NICE) system (Mierau et al., 2005)^{Error! Bookmark not defined.} was used to achieve very high Gfp expression in L. lactis NZ3900 (FIG. 4) and a human vaginal strain L. murinus KC47b (FIG. 5). Nisin is an antibiotic that is used as a food preservative. The NICE system allows regulated overproduction of a variety of interest proteins by several Gram-positive bacteria, such as L. lactis (Zhou et al., Biotech Adv 24(3):285-295, 2006).

Construction of L. lactis Bacterial Revenants:

Two Gram-positive bacterial strains, Lactococcus lactis subsp. cremoris MG1363 and Streptococcus gordonii V288, were used in this study. L. lactis MG1363 was grown at 30° C. in M17 broth supplemented with 0.5% glucose (GM17). S. gordonii V288 was grown at 37° C. in Todd-Hewitt broth.

As discussed above in Example 2, the L. lactis pretreated with glycine and the S. gordonii pretreated with penicillin and lysozyme showed best DNA delivery to mammalian cells. Without such a treatment, the rate of transfection with Gram-positive bacterial revenants was near zero (data not shown).

Therefore, these treatment methods were used for bacterial revenant production from L. lactis and S. gordonii (FIG. 9). Briefly, 20 mL overnight bacterial cultures were diluted with 100 mL pre-warmed medium and grown for 2 h at 37° C. Penicillin (100 μg/mL) and glycine (2.5% w/v) were added to S. gordonii and L. lactis cultures, respectively, and the incubation was continued for 1 h (FIG. 9B). Next, bacterial revenants were produced by adding a lethal dose of nisin (Sigma) at 2 μg/mL for 2 h to form pores in the bacterial cell membrane (FIG. 9C). The bacterial lysis was monitored by measuring the OD₆₀₀, and determination of viable cell counts. After the nisin treatment, S. gordonii was treated with lysozyme at 20 mg/mL for 1 h to further weaken the bacterial cell wall. Bacterial revenants were harvested by centrifugation (5000×g, at 4° C., 15 min), and washed 3 times with sterile deionized water and resuspended in 600 μL of HEPES-buffered saline (100 mM NaCl, 10 mM sodium acetate, 10 mM HEPES, pH 7). The washed bacterial revenants were harvested by centrifugation and stored at −80° C. as pellets in small aliquots in microtubes for future use.

Loading of L. lactis bacterial revenants: To load DNA (FIG. 9D), a frozen bacterial revenant pellet (about 10 mg) was defrosted on ice and resuspended in 200 μL of HEPES-buffered saline containing either red or green fluorescent protein plasmids (pLKV-Red2 from our own laboratory (13) or pCMV-MIR from OriGene Technologies, Inc., respectively) at 0.5 μg/μL. After CaCl₂ supplementation (25 mM) the mixture was incubated at 24° C. for 30 min with continuous gentle agitation. The loaded bacterial revenants were then harvested by centrifugation, washed once with HEPES-buffered saline, and stored at −80° C. as pellets in microtubes in small aliquots for future use.

Transfection of Human Cells by Loaded L. lactis Bacterial Revenants:

For the transfection experiment (FIG. 9E), human tongue squamous carcinoma UM1 cells were cultured in DMEM/F12 supplemented with 10% FBS, 100 U/mL penicillin and 100 μg/mL streptomycin (GIBCO) at 37° C. in a humidified incubator containing 5% CO2. The plasmid DNA-loaded bacterial revenants derived from L lactis MG1363 and S. gordonii V288 were resuspended in the DMEM/F12 medium and added to the 50-80% confluent UM1 cells cultured in 8-well chamber slides. The multiplicity of infection (MOI) was 10³ bacterial revenants/cell.

After 2-3 h of incubation at 37° C. to allow internalization of bacterial revenants, cells were washed 5 times to remove unincorporated bacterial revenants. After 48 h incubation, the expression of red and green fluorescent proteins in UM1 cells was visualized with an inverse epifluorescent microscope (Zeiss, Axioskop 40 with AxioVision Rel. 4.5 software). In the case of the green fluorescent protein expression, the blue DAPI nuclear counterstain (Thermo Scientific) was used to identify cell nuclei.

As shown in FIG. 10A, the reporter gene encoding the red fluorescent protein delivered by L. lactis MG1363 revenants was expressed well in all transfected UM1 cells. FIG. 10B shows UM1 cells transfected with the green fluorescent protein plasmid (pCMV-MIR) delivered by S. gordonii V288 revenants. FIG. 10C shows the blue DAPI nuclear counterstained cells of FIG. 10B of the same sample. By counting the red and green fluorescent cells in the microscopic photos and comparing the number with that of the blue-colored counter-stained nuclei, it appears that the transfection frequencies of this method with two different bacterial revenants both reached 100%.

The cell wall of a gram-positive bacterium may be chemically and/or physically stripped using a combination of nisin and another chemical (e.g., glycine or penicillin, as described above). Alternatively, the cell wall may be stripped using one or more chemicals and/or physical methods as described in Example 2. In some examples, the membrane may be perforated by one or more chemicals that also disrupt the cell wall. For example, nicin or lysozyme may disrupt the bacterial cell wall and perforate the membrane.

In summary, we have developed novel bacterial revenants with Gram-positive food and commensal bacteria. We used the revenants as delivery vehicles to successfully deliver bioactive molecules into mammalian cells. These delivery vehicles have several advantages over the conventional bacterial ghosts, including safety, high efficiency and capability to deliver molecules into epithelial cells. Due to the high transfection rate of this novel gene delivery system, it provides an alternative to the conventional lipid-based transfection reagents currently used for difficult-to-transfect cell types. Availability of these food and commensal BDVs will greatly facilitate research and clinical applications for mucosal vaccine, gene therapy, RNA interference and drug delivery.

### Example 4

### Alternative Method of Preparing a Gram-Positive Bacterial Revenant

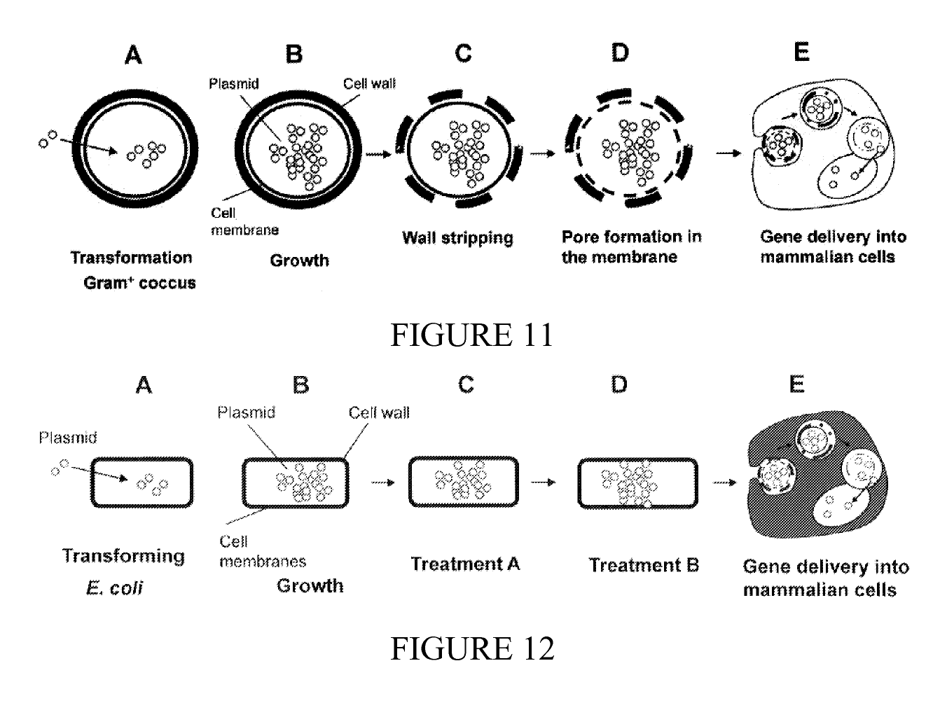

In the Example above, we demonstrated delivery of cargo (i.e., plasmids) to a mammalian cell by a Gram-positive bacterial revenant delivery vehicle loaded with the cargo after perforation of the bacterial cell membrane. An alternative method for preparing Gram-positive bacterial revenants is illustrated in FIGS. 11A-E. In this method, Gram-positive bacteria produce some or all of the cargo to be delivered (e.g., produce protein cargo, additional copies of plasmids to be delivered, etc.). Once the cargo is produced, the bacteria are treated with wall-stripping and/or membrane puncturing chemicals, resulting in bacterial revenant delivery vehicles that retain most or all of the cargo to be delivered to the targeted mammalian cells.

First, as shown in FIG. 11A, a Gram-positive bacterium is transformed with one or more plasmids (e.g., a vaccine plasmid). The transformed bacterium may be grown or maintained in culture to increase the number of copies of the plasmid and/or to allow the bacterium to produce a product (e.g., a protein) encoded by the plasmid (FIG. 11B). As shown in FIGS. 11C and 11D, the bacterium cell wall may be stripped and pores formed in the cell membrane as described above to form a bacterial revenant.

Optionally, bacterial revenants may be heated for 5 minutes at 65° C. to inactivate any remaining live bacteria. The bacterial revenants may be administered to a eukaryotic cell (e.g., a mammalian cell or tissue). Optionally, the bacterial revenants may be loaded with additional cargo after the cell membrane is perforated, as shown in FIG. 9D.

In some examples, one or more of the plasmids used to transform the bacterium may include an inducible nisin gene, and formation of pores in the cell membrane may be accomplished by inducing the production of nisin. The bacterial revenant may be added to target eukaryotic cells, such as within a topical formulation applied to a mucosal surface, to effect uptake of the bacterial revenant and delivery of the cargo to the eukaryotic cell.

### Example 5

### Gram-Negative BDVs

BDVs may also be prepared from probiotic and/or commensal, non-invasive Gram-negative bacteria. One example of a method for preparing Gram-negative bacterial vehicles is illustrated in FIGS. 12A-E. First, as shown in FIG. 12A, a Gram-negative bacterium is transformed with one or more plasmids (e.g., a vaccine plasmid). The transformed bacterium may be grown or maintained in culture to increase the number of copies of the plasmid and/or to allow the bacterium to produce a product (e.g., a protein) encoded by the plasmid (FIG. 12B). The cell wall may be at least partially stripped in one or more stages (FIG. 12C). The cell wall may be chemically stripped by treating the bacterium with one or more chemicals such as penicillin, vancomycin, a β-lactam antibiotic, a glycopeptide antibiotic, polymyxin B, polymyxin E, DL-threonine, glycine, phage lysin, and lysozyme. Because the cell wall of a gram-negative bacterium is generally thinner than the cell wall of a gram-positive bacterium, a chemical used to strip the cell wall of a gram-positive bacterium may be used at a lower concentration to strip the cell wall of a gram-negative bacterium. Alternatively, the cell wall may be physically stripped by applying one or more of electroporation, heat, or mechanical force (e.g., pressure or mixing with glass beads) to the bacterium. As still another alternative, the cell wall may be stripped using a combination of a physical method and a chemical. For example, the cell wall may be stripped by applying pressure to the bacterium and subsequently treating the bacterium with polymyxin B or another chemical.

In some examples, the membrane of the bacterium may also be perforated (FIG. 12D) as described above to form a bacterial revenant. Live bacteria, even after cell wall stripping, can interfere with the growth of cultured cells. Thus, for transfection or delivery of genes and/or other cargo to cultured cells in vitro, bacterial revenants may be used for transfection or delivery of genes and/or other cargo to cultured cells in vitro. The membrane may be perforated by one or more chemicals that also strip the cell wall. For example, the bacterium may be treated with one or more of polymyxins B and E to strip the cell wall and perforate the membrane.

In other examples, the wall-stripped bacterium may be used as a delivery vehicle without perforation of the membrane. The treated bacterium may be added to target eukaryotic cells, such as within a topical formulation applied to a mucosal surface, to effect uptake of the bacterial revenant and delivery of the cargo to the eukaryotic cell.

Chemical reagents used to weaken the cell walls and/or perforate the plasma membrane may kill about 99% of the bacteria. However, the remaining 1% of the bacteria may remain alive. Live bacteria, even after cell wall stripping, can interfere with the growth of cultured cells. Therefore, a flash heat treatment of 65° C. for 5 minutes may optionally be applied to the BDVs to inactivate the remaining live bacteria. This may be done after applying one or more the chemical reagents to the bacteria. For example, the remaining live bacteria or BDVs may be inactivated by heating and may subsequently be used for transfection or delivery of genes and/or other cargo to cultured cells in vitro. Heat inactivation of the remaining live bacteria may prevent the live bacteria from unwanted growth or interference in cell cultures.

Alternatively, membrane perforation and heat inactivation of BDVs may be omitted. Live bacteria are more resistant against gastric acid, bile salts and pancreatic enzymes in the gut than dead bacteria. Moreover, the live bacteria in the gut are non-invasive and pose no threat of harm to the host animal because they are food-grade or commensal bacteria that are non-pathogenic. Thus, live BDVs (i.e., wall-stripped but not membrane-perforated or heat inactivated) may be used for delivery of cargo in vivo, For example, live gram-negative BDVs may be used for delivery of genes, DNA, or RNAi molecules into the GI tract of an animal, such as a human. Killed (i.e., membrane-perforated and/or heat inactivated) BDVs may instead be used for delivery of cargo in vivo. For example, heat inactivated BDVs or bacterial revenants may be injected into a tissue or used in locations such as the respiratory tract, an open wound, or the oral cavity (e.g., sublingual).

### Example 6

### Systems and Methods for RNAi

RNA interference (RNAi) is a system within living cells that regulates gene expression by the activity of microRNA (miRNA) and small interfering RNA (sRNA). These small RNAs can bind to specific other RNAs to decrease protein synthesis. RNAi mediated by short interfering RNA (sRNA) is an effective approach to suppress gene expression in the mucosa. Methods have been developed using RNAi to suppress the expression of receptors of HSV (Wu et al., Cell Host Microbe 5(1):84-94, 2009) or HIV (Tamhane et al., AIDS Res Ther 5:16, 2008) as a prophylaxis against viral infections. Recently, RNAi has been used to prevent HSV-2 infection in mice by specifically suppressing the HSV receptor nectin-1 and HSV-2 UL29 with extended effect for a week (Palliser et al., Nature 439(7072):89-94, 2006; Wu et al., 2009). This is a major achievement, as most other microbicides are much shorter-lived.

While these methods are effective in animals, they are not yet feasible for human use due to safety concerns (integrating into genome), transient effect, and/or high cost. Another major challenge of the current RNAi technology is delivery efficiency. Viral vectors are effective but unsafe, while the chemically synthesized RNAs are cost-prohibitive for use in poorer, developing countries where HIV and HSV are prevalent. TransKingdom RNAi (tkRNAi) (Xiang et al., Methods Mol Biol 487:147-60, 2009; Xiang et al., 2006) is cost-efficient but it requires E. coli to express two virulence-associated genes from pathogenic bacteria: the invasin gene (Inv) from Yersinia pseudotuberculosis and the listeriolysin O gene (HlyA) from Listeria monocytogenes. This renders tkRNAi unsafe for future use in humans.

Systems, methods, and delivery vehicles (BDVs) described herein may be used to provide effective and efficient suppression of the expression of receptors in mucosal tissue and other target cells/tissues via RNAi. In at least one aspect, such systems, methods, and delivery vehicles may be used to suppress the expression of target molecules (e.g., HSV/HIV receptors) in eukaryotic tissue (e.g., in intestinal, vaginal, oral, or rectal mucosa) with improved safety, potency, cost-efficiency and effective duration. In some examples, uptake of the delivery vehicles by the mucosal epithelial cells and subsequent release of delivery vehicle cargo (e.g., shRNA and/or genes encoding shRNA) within the mucosal cells decreases viral receptor gene expression via RNAi.

Improved delivery of RNAi molecules to targeted cells provides opportunities for treatment of various diseases and conditions in vivo, as well as opportunities to analyze the role of various receptors in conditions/infections such as HSV/HIV. The novel RNAi-delivery technology described herein may be used, for example, to study HSV and HIV receptors knockdown and for viral prevention in mammals. Viral prevention strategies for HSV and HIV may include the suppression of HSV/HIV receptors in mucosa via the targeted delivery of small interfering RNA (siRNA) to HSV-2 receptors and HIV-1 receptors, respectively, using the systems, methods, and delivery vehicles described below. This technology has great potential for future human use because it will likely be safer, more potent, longer-lasting, and more cost-effective than any existing RNAi method. It is fully food grade and if any side effect is encountered it can be readily eliminated by antibiotics. The RNAi effect can be extended to life-long and costs only pennies per dose. With this technology, coitally-associated application will not be required and health disparity in the prevention of diseases and infections such as HIV will be minimized.

Delivery of Plasmid into Mucosal Cells by Noninvasive Bacteria:

As described above, we used the nisin controlled expression (NICE) system and achieved very high Gfp expression in wall-stripped L. lactis NZ3900 L. murinus KC47b (see e.g., Examples 1-2). We also constructed a novel, small-sized gene-delivery plasmid (pLKV1) capable of replicating in both Gram-negative and Gram-positive bacteria with a high copy number (about 65/cell) and a reporter plasmid pLKV-Red2. With this novel reporter plasmid, we delivered genes to Caco-2 cells with high efficiency using wall-stripped noninvasive bacteria, S. gordonii and L. lactis. We also designed and used Gram-positive bacterial revenants to deliver genes to tongue squamous carcinoma UM1 cells (Example 3).

The RNAi methods and systems described herein may be used to suppress the expression of target sequences in mucosa and other eukaryotic cells/tissues with improved safety, potency, cost-efficiency and effective duration. In some examples, BDVs loaded with cargo are taken up by mucosal epithelial cells or other eukaryotic cells, and the cargo is released within a vesicle. Thus, loaded BDVs can deliver shRNA or genes encoding shRNA to interfere with gene expression in the eukaryotic cell.

NtkRNAi may be used to produce and deliver shRNA in excessive amount into the eukaryotic cell/tissue (e.g., mucosa). NbmRNAi may be used to deliver one or more gene(s) encoding shRNA into eukaryotic cells/tissue, and the eukaryotic cells may subsequently produce the shRNA by transcription of the delivered gene(s). Optionally, NtkRNAi and NbmRNAi may be used concurrently. For example, NtkRNAi may provide fast action and short-term effect, and NbmRNAi may provide a prolonged effect. Therefore, a first group of BDVs prepared for NtkRNAi and a second group of BDVs prepared for NbmRNAi may be applied to cells/tissues together to provide a fast onset and longer duration of effect than is provided by either NtkRNAi or NbmRNAi alone.

Noninvasive TransKingdom RNA Interference (NtkRNAi):

The novel NtkRNAi described herein differs from the existing tkRNAi (Xiang et al., 2006, 2009) in both the shRNA expression plasmid and the bacterial carrier. While the existing tkRNAi bacterial carrier is invasive E. coli, the NtkRNAi bacterial carrier is a wall-stripped Gram-positive bacterial revenant or a non-invasive E. coli bacterial revenant. The bacterial revenant may be prepared, for example, essentially as described in any of the Examples described herein. In some examples, the bacterial revenant may be prepared from Lactococcus lactis, a dairy food bacterium which may safely colonize the human vagina (Todorov et al., J Basic Microbiol 46(3):226-38, 2006). In other examples, the bacterial revenant may be prepared from Lactococcus murinus and/or any other lactic acid bacterium. In still other examples, the bacterial revenant may be prepared from another non-invasive, food-grade, and/or commensal Gram-positive or Gram-negative bacterium.

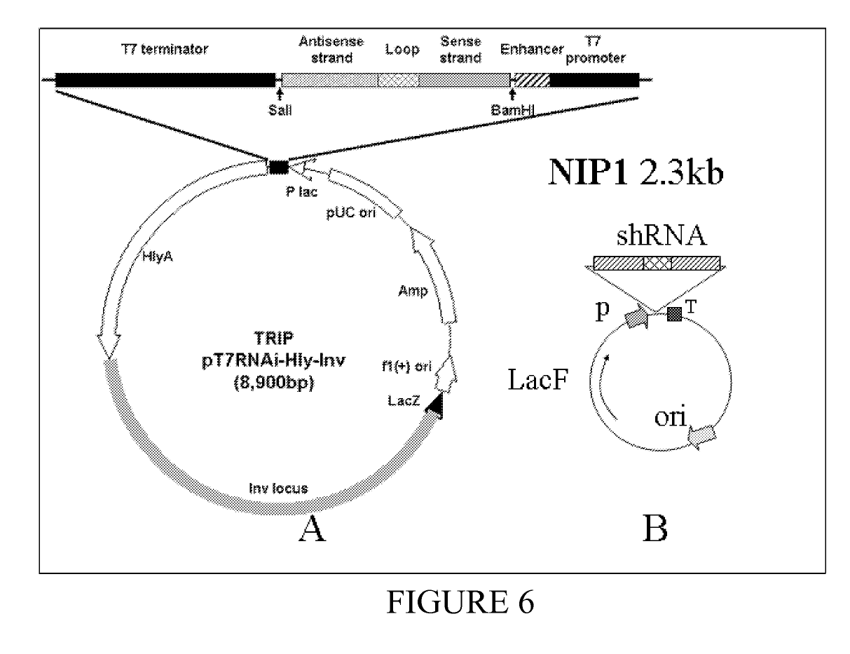

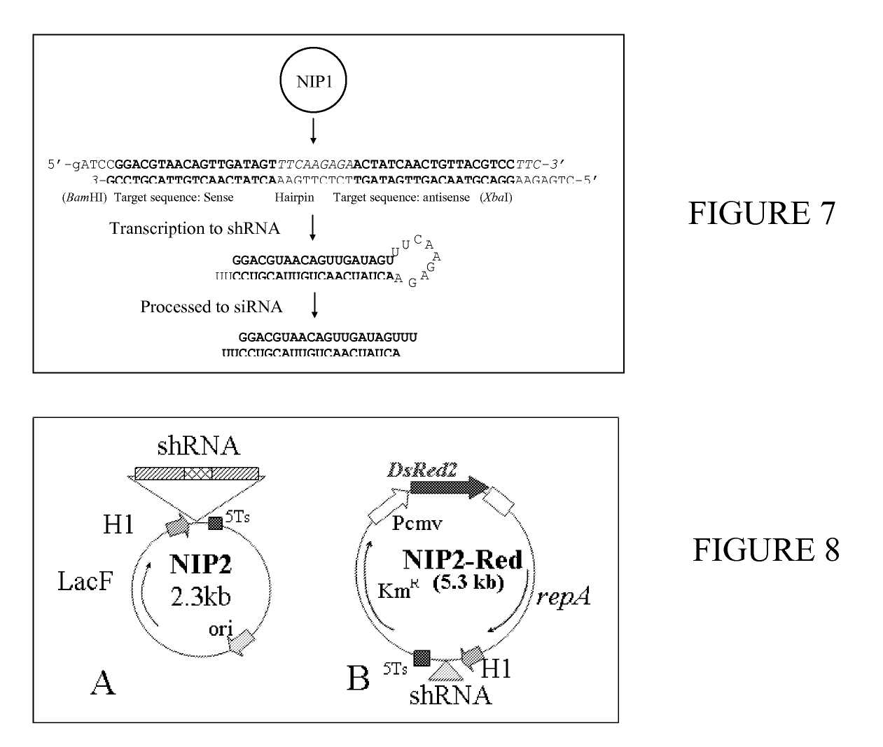

In the novel NtkRNAi system, a sense-loop-antisense sequence for production of shRNA is inserted into a transfection plasmid (e.g., into NIP1; see FIG. 6). A non-invasive bacterium is transfected with the plasmid and produces the corresponding shRNA (FIG. 7). The non-invasive bacterium is treated to weaken or strip the bacterial cell wall to form a BDV. Optionally, the plasma membrane of the BDV may be perforated to form a bacterial revenant. In some examples, additional cargo may be added to a bacterial revenant. The BDV/bacterial revenant is applied to a eukaryotic cell, which takes up the BDV by endocytosis. The eukaryotic cell then processes the shRNA into functional siRNA (FIG. 7). The siRNA may suppress or silence the expression of one or more target genes/mRNAs within the eukaryotic cell. In some examples, a reporter gene/sequence encoding a reporter may also/instead be inserted into the transfection plasmid, and the eukaryotic cell may express the delivered reporter gene/sequence.

Novel NtkRNAi Plasmid:

As shown in FIG. 6, the tkRNAi plasmid TRIP (transkingdom RNAi plasmid) contains an ampicillin resistance gene from E. coli, the invasin gene (Inv) from Yersinia pseudotuberculosis and the listeriolysin O gene (HlyA) from Listeria monocytogenes. All three of these genes are safety hazards.

In contrast, the NtkRNAi plasmid NIP1 (Non-invasive transkingdom RNAi plasmid), derived from pNZ8149 of the food-grade NICE system, is only 2.3 kb and has only a food-grade lacF or a chloramphenicol resistance gene for selection on lactose or chloramphenicol with an origin for plasmid replication in both Gram-positive and Gram-negative bacteria (FIG. 6). It has a nisin promoter and a transcriptional terminator. We have tested this plasmid with a Gfp reporter gene and it has achieved high level expression in L. lactis (FIG. 4) and Lb. murinus (FIG. 5).

shRNA Design and Cloning:

For cloning the short hairpin RNAs (shRNA), we will first test the proven siRNA nectin-1 sequence of Wu et al. (2009) to validate the NtkRNAi system (FIG. 7). Nectin-1 is the receptor of HSV-2. The RNAi plasmid will be transformed into L. lactis by electroporation. Its expression will be detected by quantitative RT-PCR with primers of Nectin-1 forward 5′-AGATGTGAAGCTCACGT GCAAAGC-3′ (SEQ ID NO: 5) and Nectin-1 reverse 5′-TTGGTGGCCTCACAGATGTAGGTT-3′ (SEQ ID NO: 6) and by Northern blot with a DNA oligo-probe or Western blot with the anti-Nectin-1 antibody. Additional shRNA may be designed targeting various receptor genes. For example, shRNA may be designed to target well established receptors, such as CD4, CCR5, CXCR4, syndecans, α4β7 integrin, and DC-SIGN for HIV and HVEM, Pilr-α for HSV. One or more shRNA or siRNA design programs, such as RNAi Explore (http://www.genelink.com/sirna/shRNAi.asp), shRNA Design Tool (http://www.ambion.com/techlib/misc/psilencer_converter.html) and/or siRNA Wizard (http://www.sirnawizard.com), may be used to design shRNA.

A scramble shRNA will be cloned into the same plasmid as the control. Groups of up to 4 different shRNA sequence designs will be cloned and tested in cultured HeLa cells for each HSV/HIV receptor gene silencing. The sequence that is the most effective against each receptor will be selected as a candidate for in vivo animal testing. If the RNAi suppression effect is not sufficient for the first group of 4 shRNA tested, a second group will be designed and tested. Additional shRNA can also be designed to target various receptor genes and can be tested in cell cultures and/or an animal model.

Noninvasive Bacteria-Mediated RNAi (NbmRNAi):

Bacteria-mediated RNAi (bmRNAi) is another approach to deliver RNA interference using naturally invasive bacteria such as Salmonella typhimurium (Zhang et al., Cancer Res 67(12):5859-64, 2007). Unlike the tkRNAi system, carrier bacteria in the bmRNAi system do not produce shRNA. Instead, they transfer shRNA expression plasmids to the host cell which then utilizes its own transcriptional machinery to produce shRNA in the nucleus. The bmRNA approach uses a eukaryotic expression promoter such as the RNA polymerase III-dependent promoters (e.g., U6 and H1) and five Ts as the terminator.

The novel NbmRNAi described in this Example differs from the existing bmRNAi in both the shRNA expression plasmid and the BDV. NbmRNAi uses non-invasive Gram-positive or Gram-negative food-grade or commensal bacteria as a delivery vehicle. In addition, the NbmRNAi plasmid replicates in the bacteria, greatly improving safety.

In the novel NbmRNAi system, a nucleic acid sequence is inserted into a transfection plasmid for transcription by a eukaryotic cell (e.g., into NIP2; see FIG. 8). A non-invasive bacterium is transfected with the plasmid and replicates the plasmid. The non-invasive bacterium is then treated to weaken or strip the bacterial cell wall to form a BDV. Optionally, the plasma membrane of the BDV may be perforated to form a bacterial revenant. In some examples, additional cargo may be added to the bacterial revenant. The BDV/bacterial revenant is applied to a eukaryotic cell, which takes up the BDV by endocytosis. The eukaryotic cell then transcribes the plasmid to produce shRNA and processes the shRNA into functional siRNA. The siRNA may suppress or silence the expression of one or more target genes/mRNAs within the eukaryotic cell. In some examples, a reporter gene/sequence encoding a reporter may be inserted into a transfection plasmid and/or non-invasive bacterium. The non-invasive bacterium or the eukaryotic cell may express the delivered reporter gene/sequence.

The BDV may be a bacterial revenant prepared from Gram-positive food-grade bacteria essentially as described above in Examples 3 and 4. In some examples, the revenant may be prepared from Lactococcus lactis. In other examples, the revenant may be prepared from Lactococcus murinus and/or any other lactic acid bacterium. In still other examples, the revenant may be prepared from another non-invasive, food-grade, and/or commensal Gram-positive or Gram-negative bacterium.

Novel NbmRNAi Plasmids:

As shown in FIG. 8, we have constructed two plasmids (NIP2 and NIP2-Red) for NbmRNAi. This approach may induce more sustained silencing because the siRNA is constantly produced by the host cell and may be more stable than siRNA produced by the bacteria and released into the host cell cytoplasm, as in tkRNAi.

shRNA Design and Cloning:

For the shRNA gene cloning, we will first test the proven siRNA nectin-1 sequence of Wu et al. (2009) to validate the NbmRNAi system (FIG. 7). The reporter plasmid NIP2-Red will be used for all laboratory and initial animal studies to monitor the success rate of the transfection. The food-grade NIP2 plasmid can be used for advanced animal studies when conditions are optimized with NIP2-Red. The shRNA design in this system is slightly different from the NtkRNAi system. A transcriptional terminator of 5 Ts is required in the tail of the shRNA sequence. shRNA design programs, such as RNAi Design Tool (www.Oligoengine.com), can be used to design shRNA sequences.

The recombinant RNAi plasmids will be transformed into L. lactis NZ3900 by electroporation. The bacterium will be used to deliver the siRNA plasmid NIP2-Red into HeLa cells. The red fluorescence expression will indicate successful transfection. The shRNA expression will be detected by quantitative RT-PCR and Northern and Western blots. Its ability to suppress Nectin-1 expression will be compared with the NtkRNAi system. A scramble shRNA will be cloned into the same plasmid as a control. Additional shRNA can also be designed to target various receptor genes and can be tested in various cell cultures and animal models as described above.

### Example 7

### Routes of Delivery to Animals

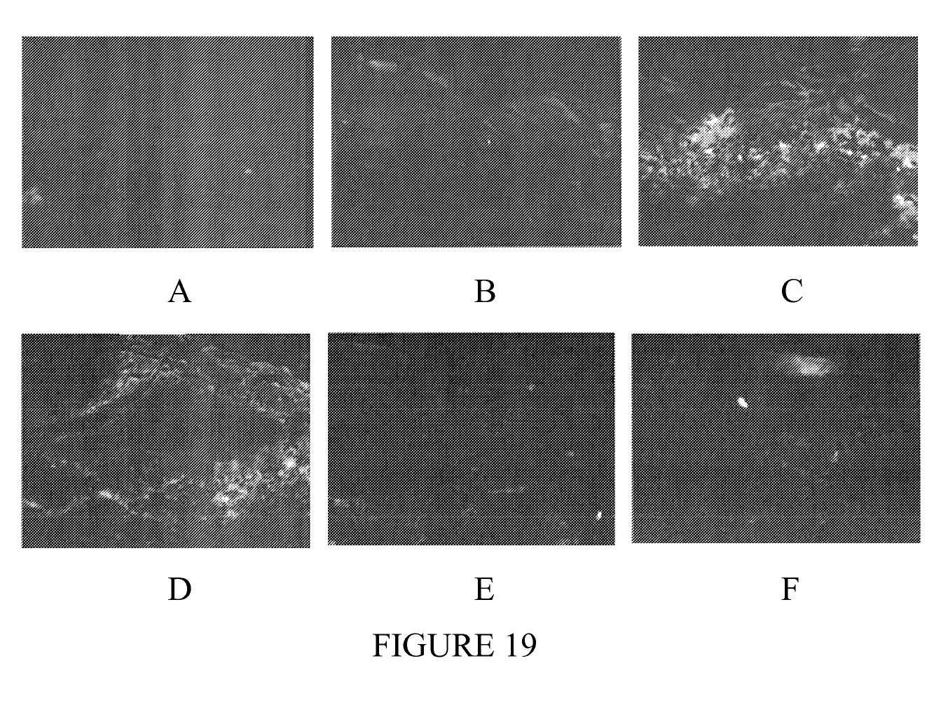

BDVs may be used to deliver cargo to eukaryotic cells in vivo or in vitro. In one embodiment, BDVs loaded with a cargo may be administered by injection into stratified squamous epithelium (e.g., in the vagina, rectum, or mouth of an animal). In other embodiments, BDVs loaded with a cargo may be administered to porous or broken epithelium without injection. For example, BDVs loaded with a cargo may be administered to intestinal epithelium, which is highly porous and can easily take up loaded BDVs. In still another embodiment, BDVs may be topically administered to a lesion, wound, or other break in an epithelial or mucosal layer (see e.g., FIGS. 19A-19F show delivery of the Red2 gene into wound tissue in a skin wound healing mouse model over a period of five days, Day 0 (FIG. 19A) to Day 5 (FIG. 19F)).

In one embodiment, BDVs may be formulated for delivery to intestinal mucosa. For example, BDVs may be microencapsulated with one or more biodegradable polymers (e.g., pectins, dextrans, or chitosan). As another example, BDVs may be delivered within enteric release capsules to achieve enteric release and bypass gastric acid damage. Alternatively, BDVs may be suspended in fluid for delivery to the colon by enema.

In other embodiments, BDVs may be formulated for delivery to the oral cavity (e.g., in a lozenge placed under the tongue, a lollipop, a spray, a gel, or an oral rinse), to the airway (e.g., as a freeze-dried powder for nasal inhalation), or to an open wound (e.g., in a cream, gel, or paste for direct application into an open wound of skin or mucosa). In still other embodiments, BDVs may be formulated for direct injection into target tissues (e.g., suspended in a fluid). In some examples, BDVs formulated for delivery to the oral cavity, airway, open wound, or by injection are fully killed (e.g., heat inactivated and/or bacterial revenants). Using fully killed BDVs may reduce or eliminate side effects that could be caused by using live bacteria in such applications.

For example, BDVs may be loaded with cargo and applied topically to an open wound or long-term unhealed lesion, such as an ulcer in the mucosa of the vagina, oral cavity, or rectum, or an unhealed skin wound of a diabetic animal. The cargo may include RNAi molecules (e.g., siRNA or shRNA), or plasmids encoding such molecules, that reduce the expression of one or more inflammatory cytokines or otherwise inhibit inflammation. The cargo may include a protein, cytokine, nucleic acid, or other molecule that promotes wound healing (e.g., fibroblast growth factor), or plasmids encoding such molecules.

As another example, the above-described NtkRNAi system may be used to produce intracellular shRNA in non-invasive bacteria. These bacteria may subsequently be treated with glycine or other wall-stripping chemical/treatment to weaken the cell wall, and the resulting BDVs may be harvested for use. Optionally, the BDVs can be resuspended into a formula with appropriate excipients suitable for the intended application. The excipients can include, for example, any one or more of a buffer, a stabilizer, a binder, thickening agents, and mucoadhesives.

Besides receptor-specific RNAi designs, the NtkRNAi and NbmRNAi systems can also be used to deliver specific RNAi against viral gene expression, such as HSV-2 UL29 (Wu et al., 2009) Although cells expressing HIV receptors, such as CCR5 and CD4, are not exposed under normal condition, they can be exposed in HSV lesions. Therefore, BDVs loaded with RNAi molecules targeting HIV and/or HSV receptors may be applied topically to HSV lesions (e.g., within the vagina), and delivery of the RNAi molecules may reduce viral gene expression.

Additional cargo, such as synthesized siRNA/plasmids, may be loaded into BDVs or bacterial revenants for delivery to mucosal or other tissues in combination with the NtkRNAi and NbmRNAi methods above. For example, synthetic nucleic acids or proteins can be added to bacterial revenants prepared for NtkRNAi or NbmRNAi to deliver both the original cargo and the additional cargo to the eukaryotic cell within a single bacterial revenant.

### Example 8

### Delivery of Vaccines

In one embodiment, BDVs may be used to deliver a vaccine to a eukaryotic cell. The vaccine may be, for example, a protein vaccine, a DNA vaccine, a DNA-protein dual vaccine, or a pseudovirus vaccine. The BDVs may be pre-loaded and/or post-loaded with any combination of vaccines for delivery to a eukaryotic cell. The vaccines delivered by the BDVs may induce the production of neutralizing antibodies within the host.

Protein Vaccines:

Protein vaccines may be constructed with the food-grade NICE system. Intracellularly-expressed vaccines in Lactobacillus (Reveneau et al., Vaccine 20:1769-1777, 2002) and Lactococcus (Cho et al., Vaccine 25:8049-8057, 2007) have been shown to induce stronger immune responses in animals. Therefore, vaccine strains that express antigenic proteins intracellularly may be constructed. Genetic transformation the bacteria may be by either natural transformation (e.g., S. gordonii) or electroporation.

It was recently reported (Mohamadzadeh et al., Proc Natl Acad Sci USA 106:4331-4336, 2009) that addition of the dendritic cell (DC)-targeting peptide (FYPSYHSTPQRP; SEQ ID NO: 7) to the C-terminus of an antigenic molecule can promote DC targeting of the antigen and even the commensal bacterium that delivers the antigen. In response to the DC-targeting peptide, dendritic cells can specifically capture the antigenic molecule and the Lactobacillus bacteria that release the antigen. Therefore, antigenic genes may optionally be modified by adding a dendritic cell-targeting peptide, such SEQ ID NO: 7, to their C-termini to promote DC-targeting.

DNA Vaccines:

The novel plasmid pLKV1 can be used to prepare DNA vaccines by cloning a target sequence into the plasmid. The plasmid can be used to transform the non-invasive bacterium (e.g., Lactococcus, Streptococcus, Lactobacillus), which may replicate the plasmid, and the bacterium can then be processed to create loaded BDVs by stripping the bacterial cell walls. Alternatively, BDVs can be created, the plasma membranes can be perforated (resulting in bacterial revenants), and the plasmids (or other cargo) can be loaded into the bacterial revenants for delivery to eukaryotic cells.

Protein-DNA Dual Vaccines:

To deliver protein and DNA vaccines simultaneously with the same bacterial vehicle, DNA vaccine plasmids can be transformed into the protein vaccine strains to construct protein-DNA dual vaccines. Between the protein and DNA vaccine plasmids, the replicons and selective markers should be different. Due to possible homologous recombination, cloning of the same HIV gene as both protein and DNA vaccines should be avoided. For example, if a protein vaccine for HIV is based on env, the corresponding DNA vaccine may be based on another sequence (e.g., gag or pol) instead of env.

Pseudovirus Vaccines:

Pseudoviruses have shown to induce potent vaccine responses in animals (Zhang et al., J Virol 78:10249-10257, 2004; Zhang et al., J Virol 78:8342-8348, 2004). A pseudovirus vaccine is a special protein-DNA dual vaccine that includes a DNA vaccine packaged inside a virus like particle (VLP) or chimeric virus like particle (CVLP). In some examples, the VLP or CVLP may be capable of pseudoinfecting a tissue (e.g., mucosal epithelium). In other examples, the VLP or CVLP may be capable of activating antigen presenting cells, which may enhance the immune response to the vaccine. Using BDVs to deliver pseudoviruses directly to the target mucosal sites may further enhance their immunogenicity. In some examples, pseudoviral vaccines may include a CVLP with two or more fused antigenic epitopes.

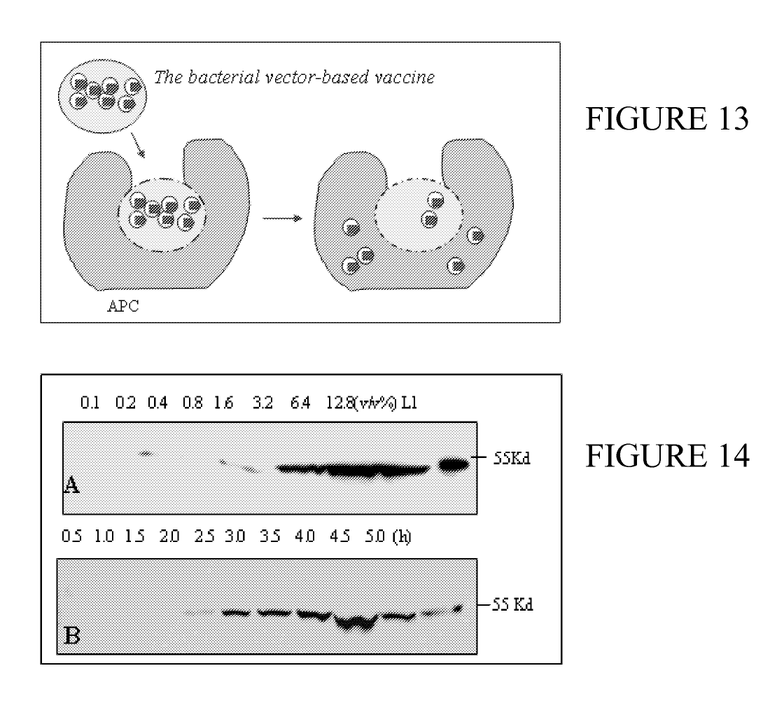

An example of a method for guided delivery of a vaccine to a eukaryotic cell by a BDV is illustrated in FIG. 13. As shown, a BDV may be loaded with a vaccine. While FIG. 13 illustrates a BDV loaded with a pseudovirus vaccine (DNA indicated by squares, capsid proteins indicated by corresponding circles), the BDV may alternatively be loaded with a DNA vaccine, a protein vaccine, another type of dual DNA-protein vaccine, or any combination thereof. Additional cargo as described above may also be loaded into the BDV. The loaded BDV may be provided to an antigen presenting cell, which may engulf the loaded BDV by phagocytosis. The vaccine may then be released within the antigen presenting cell.

FIGS. 14A and 14B illustrate dosage-dependent expression of a CVLP (BPV1 L1-gp41-2F5) in a food-grade vaccine strain of L. lactis. FIG. 14A shows the dosage-dependency of the response to inducer nisin produced by L. lactis ATCC11454, and FIG. 14B shows a time course of expression of the CVLP within the L. lactis bacteria after addition of the inducer nisin. These results show that the food-grade/commensal bacteria used to create BDVs can also be used to produce vaccines for delivery to eukaryotic cells. Therefore, using the methods disclosed herein, a non-invasive bacterium can be used to produce a vaccine or other cargo and subsequently treated with one or more chemicals to produce a BDV for efficient delivery of the cargo to eukaryotic cells.

### Example 9

### Methods for Transfection of a Eukaryotic Cell

As described above, the novel BDV technology provides highly efficient delivery of cargo to mammalian cells. The BDVs are prepared from non-invasive gram-positive or gram-negative bacteria, and may be pre-loaded and/or post-loaded with cargo for delivery to a target cell. FIG. 16 illustrates methods for delivering a gene or other cargo into a eukaryotic cell with BDVs in accordance with the present disclosure.

As shown, BDVs may optionally be pre-loaded (FIG. 16A) by transfecting the bacteria with plasmid(s)/nucleic acids. In some examples, the transfected bacteria may express at least a portion of the plasmid, resulting in the production of RNA (e.g., siRNA, shRNA, microRNA, etc.) and/or proteins, which may be delivered to the eukaryotic cell. In other examples, the bacteria may be transfected with one or more reporter plasmids/nucleic acids for confirmation of transfection. Pre-loading may include loading the bacteria with proteins, siRNA, shRNA, or other cargo for delivery to the eukaryotic cell. In some examples, the pre-loaded bacteria may be grown, cultured, or otherwise induced to replicate/express the plasmid(s) or other pre-loaded cargo for delivery to the eukaryotic cell. As shown in FIG. 16B, the pre-loading may be omitted.

The bacteria may be treated with one or more chemicals (e.g., penicillin, lysozyme, and/or glycine; see above) to strip or otherwise weaken the cell wall. For delivery vehicles that are to be post-loaded with exogenous cargo such as synthetic nucleic acids or nanoparticles, the plasma membrane may optionally be punctured/perforated by physical and/or chemical disruption (e.g., by electroporation and/or treatment with lysin or other pore-forming chemical). Alternatively, pre-loaded BDVs may be delivered to eukaryotic cells without perforating the plasma membrane. In one example, Gram-negative bacteria such as non-invasive E. coli may be pre-loaded with cargo, grown in culture, treated with one or more chemicals to strip or otherwise weaken the cell wall/capsid, and delivered to eukaryotic cells without perforating the plasma membrane or killing the bacteria. Alternatively, the plasma membrane may be perforated to kill the bacteria and/or to load exogenous cargo prior to delivery to the eukaryotic cell. Preferably, in BDVs prepared from Gram-positive bacteria, the plasma membrane is perforated prior to delivery to the eukaryotic cell.

FIG. 16C illustrates loaded BDVs. Again, while the loaded BDV is shown with a perforated plasma membrane, some loaded BDVs may have an intact/non-perforated membrane. As shown in FIG. 16D, the loaded BDVs are added to target eukaryotic cells, which engulf the BDVs via phagocytosis/endocytosis. This process may be mediated by one or more interactions between specific bacterial ligands and host (eukaryotic) cell receptors. In some examples, the weakening/stripping of the cell wall and/or perforation of the plasma membrane may expose the specific bacterial ligands for interaction with host cell receptors, and this may trigger/enhance uptake of the BDV by the host cell.

As shown in FIG. 16D, the BDV may release its cargo within a vacuole of the eukaryotic cell (e.g., an endosome, a lysosome, or a phagosome) by diffusion of the cargo across the weakened cell wall and/or perforated plasma membrane, and/or by degradation of the weakened cell wall. The cargo may be delivered to the nucleus of the eukaryotic cell. Where the cargo includes one or more nucleic acids that are expressible in eukaryotic cells, the nucleic acids may be transcribed/translated by the eukaryotic cell to produce a product such as a RNA molecule (e.g., siRNA, microRNA, shRNA, etc.) and/or a protein. In some examples, the cargo within the BDV may include one or more reporter molecules. Once the reporter molecule is within the eukaryotic cell, the eukaryotic cell may express or process the reporter molecule, providing confirmation of BDV uptake. For example, the cargo may include one or more of the reporter plasmids/genes described above, and the eukaryotic cell may become fluorescent under an epifluorescent microscope (i.e., due to expression of the red/green reporter gene) after engulfing the BDV.

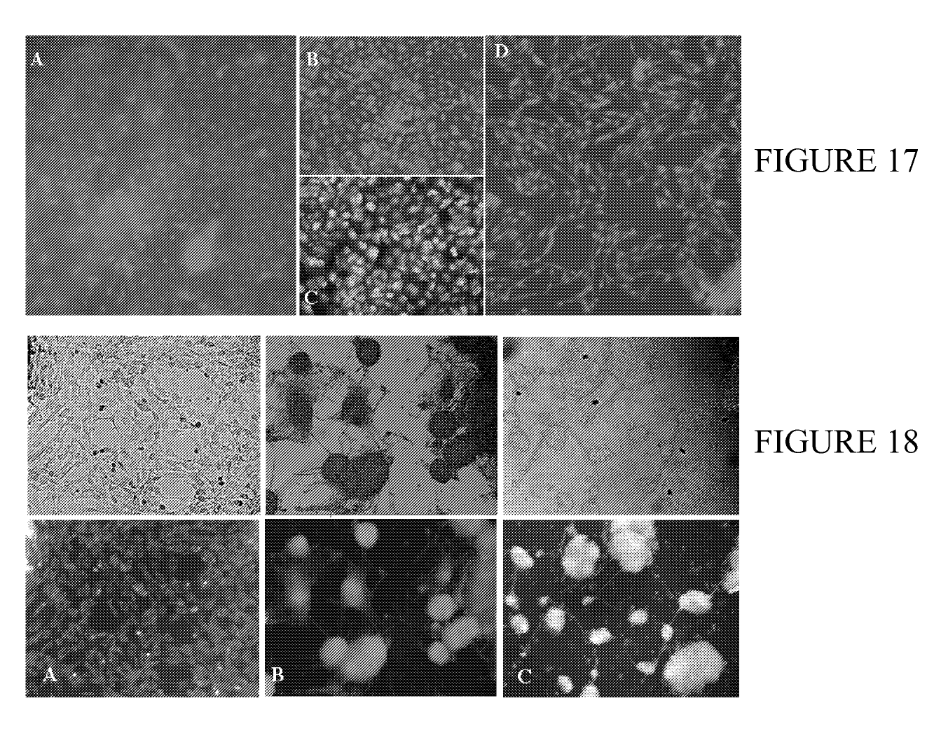

Using the above methods, we transfected epithelial cells (human colon cancer cells, human tongue squamous carcinoma cells), mesenchymal cells (human rhabdomyosarcoma muscle cells, mouse dermal fibroblasts), and neuronal cells (embryonic chicken cerebral cortex and cerebellar cells). The results are shown in FIGS. 17 and 18 as follows. FIG. 17 shows human colon cancer cells Caco-2 transfected by pLKV-Red2 via S. gordonii bacterial revenants (FIG. 17A); human tongue squamous carcinoma cell UM1 cells transfected by pLKV-Red2 via L. lactis bacterial revenants (FIG. 17B) or by pCMV-MIR via S. gordonii bacterial revenants (FIG. 17C); and human rhabdomyosarcoma muscle cell lines Rh4 (not shown) and Rh30 transfected by pLKV-Red2 via E. coli bacterial revenants (FIG. 17D). FIG. 18 shows mouse dermal fibroblasts transfected with pLKV-Red2 via L. lactis bacterial revenants (FIG. 18A), chicken cerebral cortex cells transfected with pLKV-Red2 via L. lactis bacterial revenants (FIG. 18B), and chicken cerebellar cells transfected with pCMV-MIR loaded L. lactis bacterial revenants (FIG. 18C). All these transfections achieved perfect (100%) efficiency (by counting ˜1,000 cells) with high expression of the reporter fluorescent proteins. All transfected cells were viable, and none of these cells showed significant auto-fluorescence. The same protocol was performed in other laboratories and perfect transfection was achieved in several applications.

### Example 10

### Bacterial Vehicle Delivery Reagents and Kits

Recent medical breakthroughs in gene therapy and RNA interference (RNAi) have provided hope in the treatment of many previously incurable diseases, such as diabetes (Welsh, Gene Ther 7:181-182, 2000), muscular dystrophy (Mendell et al., N Engl J Med 363:1429-37, 2010), cystic fibrosis (Davies et al., Proc Am Thorac Soc 7:408-14, 2010), cancer (Lares et al., Trends Biotechnol 28:570-9, 2010; Ozpolat et al., J Intern Med 267:44-53, 2010), and AIDS (Kitchen et al., Virology 411:260-72, 2011). However, both gene therapy and RNAi require the introduction of nucleic acids into human cells by a process called transfection, which is also a powerful tool for studying gene function and protein expression in a cell.

One of the major barriers in this field is ineffective transfection (Grigsby et al., J R Soc Interface 7(Suppl 1):S67-82, 2010). Normally, DNA or RNA molecules cannot enter mammalian cells with a negatively charged membrane. Chemicals like calcium phosphate, DEAE-dextran or cationic lipids coat the DNA or RNA, neutralizing or even creating an overall positive charge to the nucleic acid molecule to allow it to cross the membrane. Physical methods are based on totally different mechanisms to punch through the membrane to enable “forced entry” of the DNA or RNA into the cytoplasm. These include microinjection, electroporation, and biolistic particle delivery, also known as particle bombardment with a gene gun (Bonetta et. al, Nature Methods 2:875-883, 2005).

The success of gene therapy and RNAi depends on the success of transfection, so research to improve transfection efficiency has become highly active in recent years. Many commercial products have been developed to facilitate transfection. These products are largely based on artificial (chemical or physical) means, including lipids, ligands, nanoparticles, polymers, microinjection, electroporation, and particle bombardment with a gene gun (Bonetta, 2005). While some products claim to have high transfection rates, such as the X-tremeGENE of Roche and NanoJuice® Transfection Reagent Kit of Merck, such “high” transfection rates are relative to the products currently on the market. To date, none of these transfection products has reliably achieved perfect (100%) transfection.

For certain disease treatments, such as cancer gene knockdown, perfect transfection of 100% is necessary because an imperfect transfection, even as high as 99%, can leave some non-transfected cancer cells as seeds to grow back, rendering the therapy ineffective. To compensate for imperfect transfection, cells are often transfected with a plasmid containing a gene that encodes drug resistance, such as neomycin phosphotransferase (neo). After transfection, untransfected cells can be eliminated with neomycin. Although it is effective for some in vitro transfection studies, it is not practical for in vivo gene delivery to animals and humans. Therefore, technologies enabling perfect transfection have tremendous potential to advance the field of gene therapy and RNAi.

Viruses have been explored as vectors for gene delivery into mammalian cells. These include adenovirus, adeno-associated virus, herpes simplex virus and retroviruses. Lentiviruses (e.g., HIV-1) are also used because they can infect quiescent cells and integrate into the cell genome to allow stable, long-term transgene expression (Anson, Genet Vaccines Ther 2:9, 2004; Vorburger et al., Oncologist 7:46-59, 2002). Although DNA delivery by viral infection can be very efficient (50 to 100% of target cells; Goins et al., Methods Mol Biol 246:257-299, 2004), there are intrinsic biosafety issues, particularly for gene therapy. In contrast, DNA delivery into cells by transfection with non-viral methods is safer, but is usually of low efficiency (Coonrod et al., Gene Ther 4:1313-1321, 1997).

Attenuated pathogenic bacteria such as Shigella, Salmonella, Listeria and invasive E. coli have been developed as live vectors to deliver genes and anticancer therapeutics into animals and humans (Grillot-Courvalin et al., Microbe 6:115-121, 2011). As described above, bacterial ghosts (BGs) are another new technology platform based on conditional expression of the lethal lysis gene E from phage PhiX174 in Gram-negative bacteria such as Helicobacter pylori, Mannheimia haemolytica, and Salmonella enteritidis. Again, the lethal lysis gene leads to the formation of a transmembrane tunnel through the bacterial cellular envelope. Due to the high internal osmotic pressure, the cytoplasm content is expelled through the tunnel, resulting in an empty bacterial cell envelope (BG) which can be loaded with exogenous DNA. BGs can target antigen-presenting cells for vaccine delivery (Gram et al., 2007). Although current live bacterial vectors and BGs have delivered genes into cultured cells, animals and humans, they have not been developed into commercial products, nor have they achieved perfect transfection efficiency (Todorov et al, 2006; Langemann et al., Bioeng Bugs 1:326-336, 2010).

Phagocytosis is a common cellular event. Many different types of mammalian cells, even nonprofessional phagocytes, including epithelial, endothelial and mesenchymal cells, are capable of phagocytosis (Blanchette et al., PLoS ONE 4, e6056, 2009). Our novel transfection technology overcomes the limitations of the currently available transfection products and systems. In contrast to other commercial transfection products, our novel delivery method for genes and other cargo is based on a natural (biological) mechanism and uses specially killed bacterial revenants as delivery vehicles. The putative mechanism for DNA uptake by the mammalian cells is phagocytosis, which is mediated by the interaction between specific bacterial ligands and host cell receptors (Todorov et al, 2006).

The present disclosure provides methods and systems in which wall-stripped BDVs are prepared using non-invasive bacteria. Alternatively, as further described below, our BDVs may be prepared using non-invasive Gram-negative bacteria such as non-pathogenic E. coli and Listeria monocytogenes. As described above, perfect transfection of multiple different cell lines has been repeatedly achieved with the methods and BDVs disclosed herein. Therefore, these methods, systems, and BDVs may be used to develop one or more commercial transfection kits for the convenience of an end user, such as a research and/or medical facility. The commercial transfection kit(s) may deliver nucleic acids into mammalian cells with at least 90% transfection efficiency, at least 95% transfection efficiency, or perfect (100%) transfection efficiency. Persons with skill in the art will recognize that the methods and systems disclosed herein may be adjusted to accommodate variations in types and conditions of cells, growth stages, bacterial delivery vehicle dosages, and differences in cell types and laboratory settings, all of which may affect the outcomes of the delivery/transfection.

The methods and materials disclosed herein may be used for the development of commercial transfection kits that can achieve perfect transfection for multiple cell types and will have a reasonable shelf life and good stability. While the term “transfection” is used in this Example, persons with skill in the art will recognize that a variety of molecules such as proteins, peptides, antibodies, nucleic acids, therapeutic agents, and other cargo can also be introduced into eukaryotic cells in any combination via these methods/kits. Similarly, persons with reasonable skill in the art will recognize that the methods described herein may be modified using known techniques to adapt the kits/reagents to other uses. For example, one or more bacterial strains, cargo molecules, preservation methods, and/or reagents may be substituted or modified to adapt the kits/reagents for use in treatment of a disease or condition in a mammalian (e.g., human) subject, as well as for biomedical research in many areas including cell biology, biotechnology, molecular medicine, gene therapy, vaccination, and treating diseases with RNAi.

With this technology, we have achieved perfect transfection with multiple cell types, including the colon cancer Caco-2, human tongue squamous carcinoma UM1, human oral keratinocyte HOKT6, mouse dermal fibroblasts, human rhabdomyosarcoma Rh4 and Rh30 cells, and chicken neuronal cerebral cortex and cerebellar cells. Therefore, this new technology can be developed into commercial products for biomedical researchers and for use as a safer alternative to viral and pathogenic bacterial vectors for delivering gene therapy, vaccines, and RNAi. Compared with currently available transfection reagents on the market, these products offer the following advantages:
Perfect transfection
Easy shipping and storage (no need for dry ice or refrigeration)
More stable product with a longer shelf life
Inexpensive production and lower cost
Larger and more diverse cargo-packaging capacity
DNA uptake by a natural (biological) mechanism with minimal or no side effects (transfection with artificial means often kills a number of the target cells)
All natural, biodegradable materials not harmful to the cell metabolism, unlike non-degradable beads or polymers that can inhibit cell growth and metabolism.
Can deliver multiple cargo molecules (DNA/RNA/protein) simultaneously
Safe (food-grade) delivery gene therapy or RNAi to patients

Our BDV preparation/manufacturing methods are distinct from current methods for BG production in at least the following ways: (i) we produce BDVs by stripping/puncturing the bacterial cell wall (and in some cases, such as for Gram-positive bacteria, the membrane) with a combination of reagents; (ii) we can produce BDVs from any bacteria without using a cloned phage lysin; and (iii) we can produce BDVs with either Gram-negative (e.g., E. coli) or Gram-positive food/commensal bacteria that can safely deliver DNA/RNA and other cargo to humans for disease treatment and prevention.

This Example provides systems, methods, and vehicles for the development of commercial products for delivering nucleic acids into mammalian cells with perfect transfection efficiency. In one embodiment, the kit is a DIY kit that allows users to make their own BDVs (e.g., as described in the Examples above) to deliver genes that have been cloned in E. coli. In another embodiment, the kit is a BDV kit that includes one or more pre-made BDVs. The pre-made BDVs may be, for example, bacterial revenants prepared as described in any of the above Examples. This kit would allow users to load the pre-made bacterial revenants with various cargo molecules (or combinations thereof) such as DNA, RNA, antibodies, proteins, viruses or parts thereof, and/or therapeutic agents. Some kits may include reporter DNA and/or control BDVs for quality control purposes.

Methods for the development of such products as described below may include optimization of transfection conditions, standardization of transfection reagents, and/or development of prototype commercial transfection kits.

Optimization of Transfection Conditions:

Various factors that affect transfection may be optimized, including serum and other nutritional ingredients in the cell culture medium, BDV and DNA to cell ratio, cell density and growth phase, cell passage history, number of hours to measurement of reporter gene activity, and methods and reagents for BDV production and quality control.

Experimental conditions may be optimized as follows in order to maximize the success rate of a BDV-based protocol for transfection/delivery to eukaryotic cells. We will use two relatively hard-to-transfect cell lines for this. One will be an epithelial cell, the human tongue squamous carcinoma cell UM1. The other will be a mesenchymal cell, the human rhabdomyosarcoma muscle cell Rh30. Both cells transfect poorly with commercial transfection reagents (e.g., Lipofectamine of Invitrogen), but we have achieved 100% transfection with the methods disclosed herein (see e.g., Example 9).

The mechanism of gene delivery is based on cell phagocytosis, which is involved in the acquisition of nutrients for some cells and the cleaning of pathogens and cell debris for immune cells. Phagocytosis of bacteria is mediated by the interaction between specific bacterial ligands and host cell receptors. To enable phagocytosis, the cells in the culture medium must be healthy and actively growing, and the bacterial ligands must be well exposed on the surface. This requires optimizations of the cell culture medium, cell growth stages, and production of BDVs.

Optimization of Cell Culture Medium.

Many major brand cell culture media contain similar basic nutrients, including essential amino acids, vitamins, sugars and salts. To promote better cell growth, serum (such as fetal bovine serum or FBS) will be added, providing additional nutrients, hormones and growth factors. Depending on the cell types, specific growth factors will be added to the medium to stimulate the cell growth. Other supplements such as glutamine, insulin, nonessential amino acids, cholesterol and lipids can also be added to support the growth of cells that have high energy demands and synthesize large amounts of proteins and nucleic acids. Antibiotics and/or antifungals can also be added to prevent microbial contaminations. Certain antibiotics can potentiate phagocytosis against bacteria (Cuffini et al., Drugs Exp Clin Res 22:9-15, 1996). We will use a common cell culture medium such as DMEM as the basal medium to test various supplements to select for the most appropriate nutrient composition for growing the two testing cells, UM1 and Rh30.

Identification of Optimal Cell Growth Stages.

The cell growth stage is an important factor that determines the success rate of transfection experiments. Different cells may have different growth stages for optimal transfection. At the beginning, we will test only these two selected cell lines, UM1 and Rh30. To maximize transfection, we will use cells that are regularly passaged, proliferate well (best when in a log-growth phase), and are plated at a consistent density. One to two days before the transfection experiment, the cell monolayer will be trypsinized, cell concentration adjusted, and the cells plated in the chosen cell-culture vessel. The cells will be plated at six different concentrations (1-6×10⁵) in 2 ml of medium in a six-well plate overnight. In the second or third day, the cells in the six wells will achieve six different densities ranging from 50% to 95% confluency at the time of transfection. The cell growth stages for optimal transfection will be identified first with these two selected cell lines. Subsequently, the growth stages for optimal transfection of other cell lines will be identified.

Optimization of BDV Production.

BDVs developed with different methods have shown varying transfection results (Tao et al., Plasmid 65:8-14, 2011). Antibiotics that interrupt bacterial cell walls have been reported to promote phagocytosis (Cho et al., 2007). We found that besides penicillin, other bacterial wall interrupting agents, such as lysozyme, glycine and nisin, can also promote phagocytosis of the BDVs and BDV-mediated transfection efficiency. We will use four bacterial strains, S. gordonii V288, L. lactis NZ3900, E. coli JM109 and L. monocytogenes NF100 (Miner et al., Microbiol 154: 3579-3589, 2008) to produce BDVs. We will first use our existing protocols developed for these bacteria to produce the first batch of BDVs, which will be bacterial revenants, to serve as our baseline controls. Then, we will vary the conditions, including reagents, treatment time, reporter DNA loading, and dosages, to produce subsequent batches of BDVs.

Four major reagents for BDV production, penicillin, lysozyme, glycine, and nisin, will be used individually and in various combinations. Because the cell wall stripping and puncturing may not kill all the bacteria, the final BDVs will be further treated by pasteurization (65° C. for 30 min) to assure 100% killing. Different eukaryotic cell types can be sensitive to different doses of BDVs. The eukaryotic cells with more efficient phagocytosis may be stuffed to death with high dose of BDVs, while the cells with less efficient phagocytosis may require a higher dose of BDVs to achieve an efficient transfection. Therefore, the optimal BDV doses will be identified for each type of eukaryotic cells. Cell viability after transfection will be tested with trypan blue dye exclusion or alamarBlue (Invitrogen), and by continued subcultures. The BDV production will be monitored by both light and electron microscopies. Protocols that produce BDVs with the most efficient and reliable transfections will be developed and optimized.

Once we optimize the conditions for the two selected cell lines, UM1 and Rh30, we will optimize conditions for other common mammalian cells, including primary cells and transformed cells. We will make BDVs with four different bacteria because each has a different safety level and has a different range of suitable mammalian cells due to differences in their surface ligands. The invasive bacterium L. monocytogenes has been shown to trigger receptor-mediated phagocytosis of many mammalian cell types (Blanchette et al., 2009), so it may be more effective for in vitro gene delivery, although it may not be safe for in vivo human use. By having four different types of BDVs, future users will be able to choose the most appropriate BDV for their research needs.

Standardization of Transfection Reagents:

Transfection kits may include one or more reagents such as processed BDVs, DNA-loading buffers, and control reporter plasmid DNA. A standard lyophilization protocol may be developed for preservation of the processed BDVs, and lyophilized BDVs may be re-hydrated with an appropriate buffer before use. The reporter cargo DNA may be loaded into the BDVs with an optimized protocol for transfection quality control.

Reagent materials that may be standardized for commercial transfection kits include the BDVs, the cargo molecule loading buffer and the reporter plasmid DNA. In the laboratory setting, the BDVs are freshly made and stored as multiple aliquots in a deep freezer at −80° C. As a commercial product, it would be desirable for the product to be stable at ambient temperature and shippable without dry ice. Lyophilization or freeze drying is a common method for processing bio-products including BDVs (Langemann et al., 2010) for long-term and non-refrigerated storage. Therefore, we will develop a suitable lyophilization protocol for processing BDVs. The end user can simply add buffer to the lyophilized material to reconstitute the BDVs for loading cargo molecules and performing transfection/delivery to eukaryotic cells.

Lyophilization for BDVs.

To develop a lyophilization protocol, we will first test one control preloaded BDV, such as L. lactis NZ3900/pLKV-Red2. To produce BDVs, we will first treat the bacterium with cell wall weakening (e.g., glycine, penicillin and/or lysozyme) and puncturing (e.g., nisin) agents. The treated bacteria will be harvested by centrifugation and washed with buffer. Any surviving bacteria will be inactivated by pasteurization (65° C., 30 min). The killed BDVs will be re-suspended in a stabilization solution (0.5 M sucrose or trehalose) and frozen at −80° C. for 16 h. The samples will be dried with a lyophilizer for 24-48 h with water activity monitoring. The protocol that yields BDVs giving the most reliable perfect transfection will be selected for producing this particular bacterial strain. Protocols for other bacterial strains will be similarly developed and optimized.

Cargo Molecule Loading Protocol and Buffer.

A standard protocol will be developed for loading cargo molecules (DNA, RNA and/or protein) into lyophilized BDVs. First, the BDVs will be rehydrated in an appropriate buffer, such as, TE, PBS or HBS (100 mM NaCl, 10 mM NaAcetate, 10 mM Hepes, pH 7). To standardize the conditions, we will use an unloaded, blank BDV to load the eukaryotic expression reporter plasmid, pLKV-Red2, in varied conditions. These will include various ratios of BDV solid to buffer liquid and DNA to BDVs, loading times, temperatures, buffering salts and pHs, rocking speeds, etc. To track the DNA loading in real time, we will use fluorescent-dye stained DNA. After loading, we will use a fluorescent microscope to monitor the uptake of the stained DNA inside BDVs. The protocol that yields the most effective DNA loading and subsequently produces the most reliably perfect transfection will be selected as the standardized protocol. To make the future product more user-friendly, we will design a universal loading buffer suitable for all types of cargo molecules, including DNA, RNA and proteins. The buffer will contain inhibitors against DNases, RNases, and proteases, and stabilizers to extend shelf life.

We have developed an improved, proprietary lyophilization method that can maintain the viability of lyophilized lactobacilli for more than one year at high ambient temperature (32° C.) (Chang et al., The Mouth and AIDS: The Global Challenge 6th World Workshop on Oral Health and Disease, 2009). We can use this method to prepare lyophilized BDVs if the common lyophilization method does not work well. However, because the BDVs (here, bacterial revenants) are dead bacteria, viability will not be an issue. It is the structural integrity of BDVs after lyophilization that is important. Therefore, a common cryo-stabilizer, such as trehalose, may be sufficient. Because lyophilization for production and storage has been effective with BGs (Zhang et al., 2007), this method will most likely work for BDVs. However, BDVs made from different bacteria may require different optimal lyophilizing conditions. Therefore, each BDV may be optimized individually.

Commercial Transfection Kits:

Two embodiments of a commercial transfection kit are provided below: (1) a DIY BDV reagent kit for making BDVs from E. coli clones, and (2) a premade bacterial delivery kit, including 3 different premade BDVs from which users can select for loading cargo molecules. While the BDVs in the kits and products described below are bacterial revenants (i.e., BDVs with a perforated plasma membrane), persons with skill in the art will recognize that kits and products that BDVs with non-perforated plasma membranes can be developed using the same or similar methods. A universal cargo molecule loading buffer and a control reporter plasmid will be included. Appropriate packaging may also be developed to make the reagents more stable and user-friendly, and easier for shipping and storage.

DIY Delivery Vehicle Reagent Kit:

In this example, a DIY kit may include (i) a reagent mix for future users to make their own bacterial revenants with E. coli strains that contain cloned eukaryotic expression plasmids or transKingdom siRNA (Keates et al, 2008); (ii) a control plasmid; and (iii) instruction manual. We have discovered that for effective transfection with bacterial revenants, the bacterial cell wall needs to be partially stripped. This process can facilitate phagocytosis by eukaryotic cells and lysis of the engulfed bacterial revenants inside the cells. Therefore, the reagent mix may include bacterial cell wall stripping reagents: glycine, penicillin, and lysozyme in dried powders.

The control plasmid pLKV-Red2 carries a reporter gene encoding the red fluorescent protein expressed only in transfected eukaryotic cells. The plasmid will be used to transform the user's E. coli strain as a positive control for monitoring the transfection efficiency. The plasmid DNA may be dried in a screw-capped microtube.

In one example of a DIY delivery vehicle reagent kit, the reagent mix is formulated with one or more chemicals that strip the cell walls of E. coli. Optionally, the reagent mix is formulated with one or more chemicals that perforate the plasma membrane of E. coli. In this example, the DIY kit is configured to provide reagents for preparing BDVs and/or bacterial revenants from E. coli bacteria supplied by the end user.

Pre-Made Delivery Vehicle Kit:

This kit may include (i) lyophilized blank bacterial revenants made from the food bacterium L. lactis (1st choice); (ii) lyophilized blank bacterial revenants made from the commensal bacterium S. gordonii (2nd choice); (iii) lyophilized blank bacterial revenants made from the invasive bacterium Listeria monocytogenes (3rd choice); (iv) universal loading buffer; (v) pre-loaded control bacterial revenants of three different bacteria carrying pLKV-Red2; (vi) control plasmid pLKV-Red2 DNA; and (vii) instruction manual. The preloaded bacterial revenants that carry pLKV-Red2 can be used directly as a control to optimize transfection conditions and to monitor the success of the transfection experiment. To load cargo molecules, the lyophilized blank bacterial revenants may first be reconstituted with the provided universal loading buffer. The cargo molecules may then be added to the reconstituted blank bacterial revenants (see e.g., FIG. 9D). The control plasmid pLKV-Red2 can be loaded by the end user of the kit alone or simultaneously with the cargo molecule for monitoring the loading and transfection efficiency. If successful, the co-transfected cells will show both red fluorescence and a specific phenotypic change caused by the delivered cargo molecule (e.g., a gene or siRNA).

In one example, the blank bacterial revenants may be Gram-positive bacterial revenants. One or more of the blank bacterial revenants may be produced from lactic acid bacteria. Another one or more of the blank bacterial revenants may be produced from commensal or food-grade bacteria. In some examples, another one or more of the blank bacterial revenants may be produced from a bacterium that has been rendered non-invasive by genetic modification (e.g., to delete an invasion-mediating gene).

The DIY kit can be used to make fresh bacterial revenants directly from E. coli strains that carry cloned genes. These bacterial revenants can transfect eukaryotic cells with perfect efficiency. However, molecules that cannot be cloned into E. coli, such as large DNA, RNA (e.g., sRNA, shRNA, miRNA, and ncRNA), antibodies or therapeutic agents, can be loaded into the pre-made bacterial revenants for transfection. For safety reasons, the food-grade L. lactis derived bacterial revenants will be used as the primary choice. If those bacterial revenants not work in the user's application (e.g., in a particular cell line), the commensal bacterium S. gordonii-derived bacterial revenants may be used as the 2nd choice. If those bacterial revenants also do not work in the user's application, the invasive bacterium L. monocytogenes-derived bacterial revenants may be used as the third choice for in vitro study to optimize transfection conditions. After the conditions are optimized, the food-grade bacterial revenants can be re-tested in the user's application and used for gene delivery. In some examples, all the reagents in the kits may be packaged as freeze-dried powders. In other examples, one or more reagents (e.g., loading buffer) may be provided in another form, such as a liquid, solid, or gel.

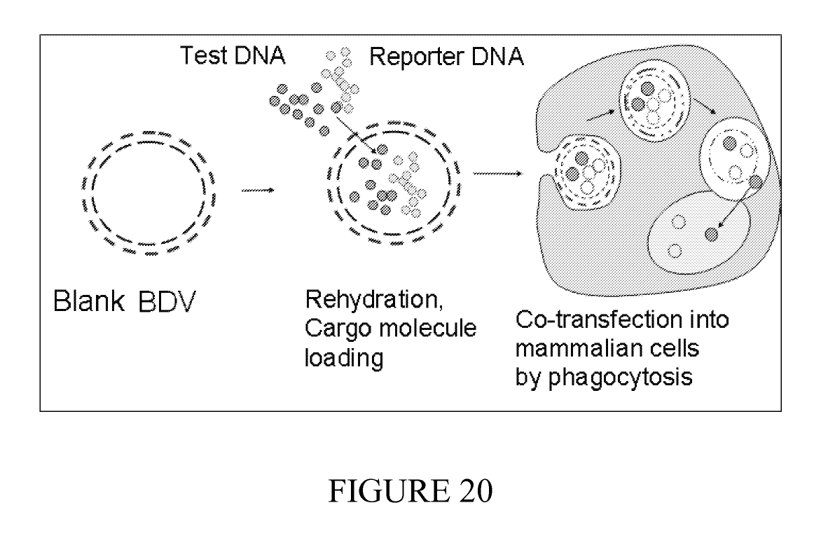

A pre-made bacterial delivery kit may be used as follows (see e.g., FIG. 20). First, the buffer is added to the dried bacterial revenants. Next, the user's cargo molecules are added to the buffer and bacterial revenants. In some examples, the buffer and the user's cargo molecules may be added simultaneously to the dried bacterial revenants. A control plasmid may also be added to the bacterial revenants. The control plasmid may be a reporter plasmid, and a portion of the control plasmid may encode a reporter molecule (e.g., Red2 or Gfp). The bacterial revenants are rehydrated by the buffer. After a pre-determined period (e.g., 5 minutes to 1 hour, 10-45 minutes, 20-40 minutes, or 30 min), the loaded bacterial revenants are ready for transfection. For example, the loaded bacterial revenants can be applied topically to a tissue or to cultured cells for uptake by the tissue/cells.

As a result of the dried powder formulation, the product stability will be maximized. To confirm the shelf lives, one or more kits may be tested periodically (e.g., a kit may be tested monthly for 12 months). In some examples, the shelf life of a DIY kit or a pre-made kit may be at least 12 months at ambient temperature. In other examples, the shelf life of the kit may be at least 24 months at temperatures up to 4° C.

Using the methods described herein, transfection kits can be developed with optimized transfection conditions and standardized transfection reagents for delivering nucleic acids into mammalian cells with perfect transfection efficiency. Such kits would be the first commercial product that can achieve perfect transfection. Unlike current transfection products on the market that are based on artificial (chemical or physical) transfection methods, the above-described kits will be based on a natural (biological) mechanism. Such kits and commercial products may significantly advance biomedical research and clinical application in gene and RNA delivery for applications such as cancer gene knockdown with RNAi and gene therapies for many diseases.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A system for delivery of molecules to a eukaryotic cell, the system comprising:
a bacterial delivery vehicle (BDV) with a plasma membrane and a weakened cell wall, wherein the BDV is produced by physical or chemical disruption of a cell wall of a non-invasive bacterium.

2. The system of claim 1, further comprising;
a cargo disposed within the BDV, wherein the cargo is delivered to the eukaryotic cell through endocytosis of the BDV by the eukaryotic cell.

3. The system of claim 1, wherein the plasma membrane is perforated and the BDV is a bacterial revenant.

4. The system of claim 1, wherein the non-invasive bacterium is a commensal bacterium, a food-grade bacterium, or a non-pathogenic bacterium.

5. The system of claim 1, wherein the non-invasive bacterium is a Gram-negative bacterium.

6. The system of claim 1, wherein the non-invasive bacterium is a Gram-positive bacterium.

7. The system of claim 2, wherein the cargo includes one or more molecules produced within the non-invasive bacterium.

8. The system of claim 1, wherein the BDV is produced by treatment of the non-invasive bacterium with one or more of lysozyme, penicillin, and glycine.

9. The system of claim 2, wherein the cargo includes one or more of a nucleic acid molecule, a protein, a virus like particle, a chimeric virus like particle, a nanoparticle, and a therapeutic drug.

10. The system of claim 2, wherein the cargo includes one or more molecules selected from the group consisting of a protein vaccine, a DNA vaccine, a protein-DNA dual vaccine, and a pseudovirus vaccine.

11. The system of claim 2, wherein the cargo includes one or more molecules selected from the group consisting of a siRNA molecule, a microRNA molecule, a shRNA molecule, and a plasmid.

12. A method for delivering a cargo into a eukaryotic cell, the method comprising administering to the eukaryotic cell a BDV, wherein the cargo is enclosed within the BDV and wherein the cargo is delivered into the eukaryotic cell through endocytosis of the non-invasive BDV by the eukaryotic cell.

13. The method of claim 12, further comprising physically or chemically disrupting an intact cell wall of a non-invasive bacterium to produce the BDV.

14. The method of claim 13, wherein the BVD is a bacterial revenant, the method further comprising physically or chemically perforating a plasma membrane of the non-invasive bacterium to produce the bacterial revenant.

15. The method of claim 13, further comprising loading the cargo into the non-invasive bacterium by electroporation.

16. The method of claim 14, further comprising loading the cargo into the bacterial revenant.

17. The method of claim 15, wherein the cargo comprises one or more molecules selected from the group consisting of a nucleic acid molecule, a protein, a virus like particle, a chimeric virus like particle, a DNA vaccine, a protein vaccine, and a therapeutic drug.

18. A plasmid vector comprising:
an origin of replication;
a kanamycin resistance marker; and
a eukaryotic gene expression cassette,
wherein the plasmid vector is less than 4.5 kb in size, and wherein the origin of replication and the kanamycin resistance marker are expressible in both Gram-positive and Gram-negative bacteria.

19. A reporter plasmid comprising:
an origin of replication;
a kanamycin resistance marker;
a eukaryotic gene expression cassette; and
a reporter sequence encoding a reporter molecule,
wherein the reporter plasmid is less than 5.2 kb in size, the origin of replication and the kanamycin resistance marker are expressible in both Gram-positive and Gram-negative bacteria, and the reporter sequence is expressible in eukaryotic cells and not expressible in Gram-positive bacteria or Gram-negative bacteria.

20. A pharmaceutical compound comprising one or more BDVs or bacterial revenants admixed with an excipient.

21. The pharmaceutical compound of claim 20, further comprising a cargo disposed within the one or more BDVs.

22. The pharmaceutical compound of claim 21, wherein the cargo includes one or more of a nucleic acid molecule, a protein, a virus like particle, a chimeric virus like particle, a nanoparticle, or a therapeutic drug.

23. The pharmaceutical compound of claim 21, wherein the cargo includes one or more molecules selected from the group consisting of a protein vaccine, a DNA vaccine, a protein-DNA dual vaccine, and a pseudovirus vaccine.

24. The pharmaceutical compound of claim 21, wherein the cargo includes one or more molecules selected from the group consisting of a siRNA molecule, a microRNA molecule, a shRNA molecule, and a plasmid.

25. The pharmaceutical compound of claim 20, wherein the excipient is formulated for application to an epithelial or mucosal tissue.

26. The pharmaceutical compound of claim 20, wherein the excipient includes one or more of a buffer, a stabilizer, a binder, a thickening agent, or a mucoadhesive.

27. The pharmaceutical compound of claim 20, wherein the pharmaceutical compound is disposed on or within a food, an injectable liquid, a topical formulation, or a suppository.

28. A kit for delivery of a cargo to a eukaryotic cell, the kit comprising:
one or more chemicals configured to weaken bacterial cell walls, wherein application of the one or more chemicals to a non-invasive bacterium results in the formation of a BDV; and
a control plasmid, wherein a portion of the control plasmid encodes a reporter molecule.

29. The kit of claim 28, wherein the portion of the control plasmid is expressible in eukaryotic cells and is not expressible in Gram-positive or Gram-negative bacterial cells.

30. The kit of claim 28, wherein at least one of the one or more chemicals is selected from the group consisting of glycine, lysozyme, nisin, and penicillin.

31. A kit for delivery of a cargo to a eukaryotic cell, the kit comprising:
a plurality of bacterial revenants; and
a control plasmid, wherein a portion of the control plasmid encodes a reporter molecule.

32. The kit of claim 31, wherein the plurality of bacterial revenants includes a first group of bacterial revenants prepared from a first species of bacteria and a second group of bacterial revenants prepared from a second species of bacteria.

33. The kit of claim 32, wherein the plurality of bacterial revenants further includes a third group of bacterial revenants prepared from a third species of bacteria different from the first and second species.

34. The kit of claim 31, wherein some of the bacterial revenants of the plurality contain a copy of the control plasmid.

35. The kit of claim 31, wherein the bacterial revenants of the plurality are supplied in a lyophilized form.

36. The kit of claim 31, further including a loading buffer.
